SEQUENCE LISTING (358 sequences)

SEQ ID NO: 1 - DNA, 20 bp, Artificial Sequence
  Antisense Oligonucleotide
tccgtcatcgctcctcaggg

SEQ ID NO: 2 - DNA, 20 bp, Artificial Sequence
  Antisense Oligonucleotide
gtgcgcgcgagcccgaaatc

SEQ ID NO: 3 - DNA, 20 bp, Artificial Sequence
  Antisense Oligonucleotide
atgcattctgcccccaagga

SEQ ID NO: 4 - DNA, 2271 bp, H. sapiens
  CDS (185)...(1138)
aagcttggtaccgagctcggatccactactcgacccacgcgtccgcgcgccccaggagccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtacctttttttttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaaaaaaaaaaaaagggcggccgctctagagggccctattctatag

SEQ ID NO: 5 - DNA, 30 bp, Artificial Sequence
  PCR Primer
cctagctacagagtatcttcaactaatggt

SEQ ID NO: 6 - DNA, 21 bp, Artificial Sequence
  PCR Primer
tggtgcttcctcctttcatca

SEQ ID NO: 7 - DNA, 31 bp, Artificial Sequence
  PCR Probe
cgtcactaaaaccagcatcaaaatcccaagt

SEQ ID NO: 8 - DNA, 19 bp, Artificial Sequence
  PCR Primer
gaaggtgaaggtcggagtc

SEQ ID NO: 9 - DNA, 20 bp, Artificial Sequence
  PCR Primer
gaagatggtgatgggatttc

SEQ ID NO: 10 - DNA, 20 bp, Artificial Sequence
  PCR Probe
caagcttcccgttctcagcc

SEQ ID NO: 11 - DNA, 2237 bp, M. musculus
  CDS (142)...(1092)
cccacgtcccggggagccactgccaggacctttgtgaaccggtcggggcgggggccgtggcggagtctgctcggcggtgggtggcccgagaagggagagaacgatcgcggagcagggcgcccgaactccgggcgccgcgccatgcgccgggccagccgagactacggcaagtacctgcgcagctcggaagagatgggcagcggccccggcgtcccacacgaaggtccgctgcaccccgcgccttctgcaccggctccggcgccgccacccgccgcctcccgctccatgttcctggccctcctggggctgggactgggccaggtggtctgcagcatcgctctgttcctgtactttcgagcgcagatggatcctaacagaatatcagaagacagcactcactgcttttatagaatcctgagactccatgaaaacgcaggtttgcaggactcgactctggagagtgaagacacactacctgactcctgcaggaggatgaaacaagcctttcagggggccgtgcagaaggaactgcaacacattgtggggccacagcgcttctcaggagctccagctatgatggaaggctcatggttggatgtggcccagcgaggcaagcctgaggcccagccatttgcacacctcaccatcaatgctgccagcatcccatcgggttcccataaagtcactctgtcctcttggtaccacgatcgaggctgggccaagatctctaacatgacgttaagcaacggaaaactaagggttaaccaagatggcttctattacctgtacgccaacatttgctttcggcatcatgaaacatcgggaagcgtacctacagactatcttcagctgatggtgtatgtcgttaaaaccagcatcaaaatcccaagttctcataacctgatgaaaggagggagcacgaaaaactggtcgggcaattctgaattccacttttattccataaatgttgggggatttttcaagctccgagctggtgaagaaattagcattcaggtgtccaacccttccctgctggatccggatcaagatgcgacgtactttggggctttcaaagttcaggacatagactgagactcatttcgtggaacattagcatggatgtcctagatgtttggaaacttcttaaaaaatggatgatgtctatacatgtgtaagactactaagagacatggcccacggtgtatgaaactcacagccctctctcttgagccctgtacaggttgtgtatatgtaaagtccataggtgatgttagattcatggtgattacacaacggttttacaattttgtaatgatttcctagaattgaaccagattgggagaggtattccgatgcttatgaaaaacttacacgtgagctatggaagggggtcacagtctctggtctaacccctggacatgtgccactgagaaccttgaaattaagaggatgccatgtcattgcatagaaatgatagtgtgaagggttaagttcttttgaattgttacattgcgctgggacctgcaaataagttctttttttctaatgaggagaaaaatatatgtatttttatataatgtctaaagttatatttcaggtgtaatgttttctgtgcaaagttttgtaaattatatttgtgctatagtatttgattcaaaatatttaaaaatgtctcactgttgacatatttaatgttttaaatgtacagatgtatttaactggtgcactttgtaattcccctgaaggtactcgtagctaagggggcagaatactgtttctggtgaccacatgtagtttatttctttattctttttaacttaatagagtcttcagacttgtcaaaactatgcaagcaaaataaataaataaaaataaaatgaataccttgaataataagtaggatgttggtcaccaggtgcctttcaaatttagaagctaattgactttaggagctgacatagccaaaaaggaacataataggctactgaaatctgtcaggagtatttatgcaattattgaacaggtgtctttttttacaagagctacaaattgtaaattttggtttcttttttttcccatagaaaatgtactatagtttatcagccaaaaaacaatccactttttaatttagtgaaagttattttattatactgtacaataaaagcattgtctctgaatgttaattttttggtacaaaaaataaatttgtacgaaaaaaaaaaaaaaaaaaaaaaaaa

SEQ ID NO: 12 - DNA, 19 bp, Artificial Sequence
  PCR Primer
tgcagcatcgctctgttcc

SEQ ID NO: 13 - DNA, 23 bp, Artificial Sequence
  PCR Primer
aagcagtgagtgctgtcttctga

SEQ ID NO: 14 - DNA, 28 bp, Artificial Sequence
  PCR Probe
tttcgagcgcagatggatcctaacagaa

SEQ ID NO: 15 - DNA, 20 bp, Artificial Sequence
  PCR Primer
ggcaaattcaacggcacagt

SEQ ID NO: 16 - DNA, 20 bp, Artificial Sequence
  PCR Primer
gggtctcgctcctggaagat

SEQ ID NO: 17 - DNA, 27 bp, Artificial Sequence
  PCR Probe
aaggccgagaatgggaagcttgtcatc

SEQ ID NO: 18 - DNA, 957 bp, R. norvegicus
  CDS (1)...(957)
atgcgccgggccaaccgagactacggcaagtacctgcgcggctcggaggagatgggcagttgccctggcgtcccacacgagggtccgctgcatcccgcgccttcagcaccggctccagcgccgccccccgccgcctcccgcttcatgttcctggcgctcctggggctgggactgggtcaggtggtctgcagcatcgctctgttcctgtactttcgagcgcagatggatcctaacagaatatcagaagacagcacgcgctgcttctacagaattctgagactccgtgaaaatacaggtttgcaggactcgactctggagagcgaagacacagaagcactacctgactcatgcaggagaatgaaacaagcctttcaaggggccgtgcaaagggaattacaacacattgtggggccacagcgcttctcaggagttccagctatgatggaaggttcgtggctcgatgtggcccggcggggcaagcctgaggctcagccgtttgctcacctcaccatcaatgctgccgacatcccatcgggttcccataaagtcagtctgtcctcttggtaccatgatcgaggctgggccaagatctctaacatgacgttaagcaacggaaaactaagggttaaccaagatggcttctattacctgtacgccaacatttgcttcaggcatcatgaaacctcagggagcgtacctgcggactatcttcagctgatggtatatgtcgttaaaaccagcatcaaaatcccaagttcgcataacctgatgaaaggggggagcactaagaactggtcagggaattctgaattccacttttattccataaacgttggaggatttttcaagctccgggctggtgaggaaattagcgtccaggtgtccaacccttccctgttggatccggatcaagatgcgacgtactttggggctttcaaagttcaagacatagactga

SEQ ID NO: 19 - DNA, 25 bp, Artificial Sequence
  PCR Primer
tttattccataaacgttggaggatt

SEQ ID NO: 20 - DNA, 21 bp, Artificial Sequence
  PCR Primer
ttggacacctggacgctaatt

SEQ ID NO: 21 - DNA, 22 bp, Artificial Sequence
  PCR Probe
ttcaagctccgggctggtgagg

SEQ ID NO: 22 - DNA, 23 bp, Artificial Sequence
  PCR Primer
tgttctagagacagccgcatctt

SEQ ID NO: 23 - DNA, 21 bp, Artificial Sequence
  PCR Primer
caccgaccttcaccatcttgt

SEQ ID NO: 24 - DNA, 24 bp, Artificial Sequence
  PCR Probe
ttgtgcagtgccagcctcgtctca

SEQ ID NO: 25 - DNA, 20 bp, Artificial Sequence
  Antisense Oligonucleotide
ccgagctcggtaccaagctt

SEQ ID NO: 26 - DNA, 20 bp, Artificial Sequence
  Antisense Oligonucleotide
cggacgcgtgggtcgagtag

SEQ ID NO: 27 - DNA, 20 bp, Artificial Sequence
  Antisense Oligonucleotide
cggccaactccggaggctgc

SEQ ID NO: 28 - DNA, 20 bp, Artificial Sequence
  Antisense Oligonucleotide
ctcccgctccctccccttct

SEQ ID NO: 29 - DNA, 20 bp, Artificial Sequence
  Antisense Oligonucleotide
ctctcgcttcggagctctcc

SEQ ID NO: 30 - DNA, 20 bp, Artificial Sequence
  Antisense Oligonucleotide
tggcgctcggccctctcgct

SEQ ID NO: 31 - DNA, 20 bp, Artificial Sequence
  Antisense Oligonucleotide
cgcggcgcatggcgctcggc

SEQ ID NO: 32 - DNA, 20 bp, Artificial Sequence
  Antisense Oligonucleotide
ctctgctggcgcggcgcatg

SEQ ID NO: 33 - DNA, 20 bp, Artificial Sequence
  Antisense Oligonucleotide
gagccacgcaggtacttggt

SEQ ID NO: 34 - DNA, 20 bp, Artificial Sequence
  Antisense Oligonucleotide
ctggtgcggcgcaggcggcg

SEQ ID NO: 35 - DNA, 20 bp, Artificial Sequence
  Antisense Oligonucleotide
cccagccccaggagggccac

SEQ ID NO: 36 - DNA, 20 bp, Artificial Sequence
  Antisense Oligonucleotide
acgctgcagacaacctggcc

SEQ ID NO: 37 - DNA, 20 bp, Artificial Sequence
  Antisense Oligonucleotide
cgctctgaaatagaagaaca

SEQ ID NO: 38 - DNA, 20 bp, Artificial Sequence
  Antisense Oligonucleotide
tctgatattctattaggatc

SEQ ID NO: 39 - DNA, 20 bp, Artificial Sequence
  Antisense Oligonucleotide
aaattctataaatgcagtga

SEQ ID NO: 40 - DNA, 20 bp, Artificial Sequence
  Antisense Oligonucleotide
ttcatggagtctcaaaattc

SEQ ID NO: 41 - DNA, 20 bp, Artificial Sequence
  Antisense Oligonucleotide
gaaaatctgcattttcatgg

SEQ ID NO: 42 - DNA, 20 bp, Artificial Sequence
  Antisense Oligonucleotide
agagttgtgtcttgaaaatc

SEQ ID NO: 43 - DNA, 20 bp, Artificial Sequence
  Antisense Oligonucleotide
cttgactctccagagttgtg

SEQ ID NO: 44 - DNA, 20 bp, Artificial Sequence
  Antisense Oligonucleotide
gaatcaggtattaattttgt

SEQ ID NO: 45 - DNA, 20 bp, Artificial Sequence
  Antisense Oligonucleotide
tctcctacatgaatcaggta

SEQ ID NO: 46 - DNA, 20 bp, Artificial Sequence
  Antisense Oligonucleotide
aggcctgtttaattctccta

SEQ ID NO: 47 - DNA, 20 bp, Artificial Sequence
  Antisense Oligonucleotide
ttccttttgcacagctcctt

SEQ ID NO: 48 - DNA, 20 bp, Artificial Sequence
  Antisense Oligonucleotide
gatccaacgatatgttgtaa

SEQ ID NO: 49 - DNA, 20 bp, Artificial Sequence
  Antisense Oligonucleotide
ctttctctgctctgatgtgc

SEQ ID NO: 50 - DNA, 20 bp, Artificial Sequence
  Antisense Oligonucleotide
taaccatgagccatccacca

SEQ ID NO: 51 - DNA, 20 bp, Artificial Sequence
  Antisense Oligonucleotide
gcttgctcctcttggccaga

SEQ ID NO: 52 - DNA, 20 bp, Artificial Sequence
  Antisense Oligonucleotide
atgagcaaaaggctgagctt

SEQ ID NO: 53 - DNA, 20 bp, Artificial Sequence
  Antisense Oligonucleotide
gatgtcggtggcattaatag

SEQ ID NO: 54 - DNA, 20 bp, Artificial Sequence
  Antisense Oligonucleotide
actcactttatgggaaccag

SEQ ID NO: 55 - DNA, 20 bp, Artificial Sequence
  Antisense Oligonucleotide
cgatcatggtaccaagagga

SEQ ID NO: 56 - DNA, 20 bp, Artificial Sequence
  Antisense Oligonucleotide
tgttggagatcttggcccaa

SEQ ID NO: 57 - DNA, 20 bp, Artificial Sequence
  Antisense Oligonucleotide
agttttccattgctaaaagt

SEQ ID NO: 58 - DNA, 20 bp, Artificial Sequence
  Antisense Oligonucleotide
ttaactattagttttccatt

SEQ ID NO: 59 - DNA, 20 bp, Artificial Sequence
  Antisense Oligonucleotide
aaaagccatcctgattaact

SEQ ID NO: 60 - DNA, 20 bp, Artificial Sequence
  Antisense Oligonucleotide
caaatgttggcatacaggta

SEQ ID NO: 61 - DNA, 20 bp, Artificial Sequence
  Antisense Oligonucleotide
tgaagtttcatgatgtcgaa

SEQ ID NO: 62 - DNA, 20 bp, Artificial Sequence
  Antisense Oligonucleotide
gatactctgtagctaggtct

SEQ ID NO: 63 - DNA, 20 bp, Artificial Sequence
  Antisense Oligonucleotide
agtgacgtacaccattagtt

SEQ ID NO: 64 - DNA, 20 bp, Artificial Sequence
  Antisense Oligonucleotide
aacttgggattttgatgctg

SEQ ID NO: 65 - DNA, 20 bp, Artificial Sequence
  Antisense Oligonucleotide
cctcctttcatcagggtatg

SEQ ID NO: 66 - DNA, 20 bp, Artificial Sequence
  Antisense Oligonucleotide
ttccctgaccaatacttggt

SEQ ID NO: 67 - DNA, 20 bp, Artificial Sequence
  Antisense Oligonucleotide
aatccaccaacgtttatgga

SEQ ID NO: 68 - DNA, 20 bp, Artificial Sequence
  Antisense Oligonucleotide
gacctcgatgctgatttcct

SEQ ID NO: 69 - DNA, 20 bp, Artificial Sequence
  Antisense Oligonucleotide
tgttgcatcctgatccggat

SEQ ID NO: 70 - DNA, 20 bp, Artificial Sequence
  Antisense Oligonucleotide
tcgaactttaaaagccccaa

SEQ ID NO: 71 - DNA, 20 bp, Artificial Sequence
  Antisense Oligonucleotide
catccaggaaatacataaca

SEQ ID NO: 72 - DNA, 20 bp, Artificial Sequence
  Antisense Oligonucleotide
tatacatctttcttggcttg

SEQ ID NO: 73 - DNA, 20 bp, Artificial Sequence
  Antisense Oligonucleotide
atgcctcttagtagtctcac

SEQ ID NO: 74 - DNA, 20 bp, Artificial Sequence
  Antisense Oligonucleotide
aggtcaagagcatggatact

SEQ ID NO: 75 - DNA, 20 bp, Artificial Sequence
  Antisense Oligonucleotide
ctgtaaatacgcgtgttctc

SEQ ID NO: 76 - DNA, 20 bp, Artificial Sequence
  Antisense Oligonucleotide
atgagtctaacatctcccac

SEQ ID NO: 77 - DNA, 20 bp, Artificial Sequence
  Antisense Oligonucleotide
aattcattacaaaatttaaa

SEQ ID NO: 78 - DNA, 20 bp, Artificial Sequence
  Antisense Oligonucleotide
ccaatctggtttaattctag

SEQ ID NO: 79 - DNA, 20 bp, Artificial Sequence
  Antisense Oligonucleotide
ataaggtcaacccgtaattg

SEQ ID NO: 80 - DNA, 20 bp, Artificial Sequence
  Antisense Oligonucleotide
catagcccacatgcagtttc

SEQ ID NO: 81 - DNA, 20 bp, Artificial Sequence
  Antisense Oligonucleotide
catgaccagggaccaacccc

SEQ ID NO: 82 - DNA, 20 bp, Artificial Sequence
  Antisense Oligonucleotide
ctagatgacaccctctccac

SEQ ID NO: 83 - DNA, 20 bp, Artificial Sequence
  Antisense Oligonucleotide
ttcagatgatccttcaattg

SEQ ID NO: 84 - DNA, 20 bp, Artificial Sequence
  Antisense Oligonucleotide
acaattcaaaagaatttgcc

SEQ ID NO: 85 - DNA, 20 bp, Artificial Sequence
  Antisense Oligonucleotide
gcaggttccagcatgatgta

SEQ ID NO: 86 - DNA, 20 bp, Artificial Sequence
  Antisense Oligonucleotide
atataactttagatattata

SEQ ID NO: 87 - DNA, 20 bp, Artificial Sequence
  Antisense Oligonucleotide
tttgcaaagaaaacattaca

SEQ ID NO: 88 - DNA, 20 bp, Artificial Sequence
  Antisense Oligonucleotide
agcacaaatataatttacaa

SEQ ID NO: 89 - DNA, 20 bp, Artificial Sequence
  Antisense Oligonucleotide
ttaaatattttgaatcaaat

SEQ ID NO: 90 - DNA, 20 bp, Artificial Sequence
  Antisense Oligonucleotide
acatttaaaacattaaatat

SEQ ID NO: 91 - DNA, 20 bp, Artificial Sequence
  Antisense Oligonucleotide
tgcaccagttaaatatgtct

SEQ ID NO: 92 - DNA, 20 bp, Artificial Sequence
  Antisense Oligonucleotide
ttttccccagggaatttaca

SEQ ID NO: 93 - DNA, 20 bp, Artificial Sequence
  Antisense Oligonucleotide
gatattaggaaacaacattt

SEQ ID NO: 94 - DNA, 20 bp, Artificial Sequence
  Antisense Oligonucleotide
gaagaaatatactgcatttg

SEQ ID NO: 95 - DNA, 20 bp, Artificial Sequence
  Antisense Oligonucleotide
aagaacgaagaaatatactg

SEQ ID NO: 96 - DNA, 20 bp, Artificial Sequence
  Antisense Oligonucleotide
tattattcaaggcatccatt

SEQ ID NO: 97 - DNA, 20 bp, Artificial Sequence
  Antisense Oligonucleotide
cctggtggccaacatcctgc

SEQ ID NO: 98 - DNA, 20 bp, Artificial Sequence
  Antisense Oligonucleotide
tagtttctaaatttgaaagg

SEQ ID NO: 99 - DNA, 20 bp, Artificial Sequence
  Antisense Oligonucleotide
caatgtcagctttctaaagt

SEQ ID NO: 100 - DNA, 20 bp, Artificial Sequence
  Antisense Oligonucleotide
ttgacagatttcagtggccc

SEQ ID NO: 101 - DNA, 20 bp, Artificial Sequence
  Antisense Oligonucleotide
gttcaacaattatataacta

SEQ ID NO: 102 - DNA, 20 bp, Artificial Sequence
  Antisense Oligonucleotide
actaataacttttctatttt

SEQ ID NO: 103 - DNA, 20 bp, Artificial Sequence
  Antisense Oligonucleotide
aacatttactaaattaaaat

SEQ ID NO: 104 - DNA, 20 bp, Artificial Sequence
  Antisense Oligonucleotide
aacattcaaaggcaatgttt

SEQ ID NO: 105 - DNA, 20 bp, Artificial Sequence
  Antisense Oligonucleotide
atccgagctcggtaccaagc

SEQ ID NO: 106 - DNA, 20 bp, Artificial Sequence
  Antisense Oligonucleotide
gcggacgcgtgggtcgagta

SEQ ID NO: 107 - DNA, 20 bp, Artificial Sequence
  Antisense Oligonucleotide
agcccggctttggctcctgg

SEQ ID NO: 108 - DNA, 20 bp, Artificial Sequence
  Antisense Oligonucleotide
ccggaggctgcggcggagcc

SEQ ID NO: 109 - DNA, 20 bp, Artificial Sequence
  Antisense Oligonucleotide
ggccaactccggaggctgcg

SEQ ID NO: 110 - DNA, 20 bp, Artificial Sequence
  Antisense Oligonucleotide
cgcttcggagctctcctccc

SEQ ID NO: 111 - DNA, 20 bp, Artificial Sequence
  Antisense Oligonucleotide
atggcgctcggccctctcgc

SEQ ID NO: 112 - DNA, 20 bp, Artificial Sequence
  Antisense Oligonucleotide
gcatggcgctcggccctctc

SEQ ID NO: 113 - DNA, 20 bp, Artificial Sequence
  Antisense Oligonucleotide
cggcgcatggcgctcggccc

SEQ ID NO: 114 - DNA, 20 bp, Artificial Sequence
  Antisense Oligonucleotide
tctctgctggcgcggcgcat

SEQ ID NO: 115 - DNA, 20 bp, Artificial Sequence
  Antisense Oligonucleotide
catctcctccgagccacgca

SEQ ID NO: 116 - DNA, 20 bp, Artificial Sequence
  Antisense Oligonucleotide
gggctggtgcggcgcaggcg

SEQ ID NO: 117 - DNA, 20 bp, Artificial Sequence
  Antisense Oligonucleotide
gaacatggagcgggaggcgg

SEQ ID NO: 118 - DNA, 20 bp, Artificial Sequence
  Antisense Oligonucleotide
tggcccagccccagccccag

SEQ ID NO: 119 - DNA, 20 bp, Artificial Sequence
  Antisense Oligonucleotide
cctggcccagccccagcccc

SEQ ID NO: 120 - DNA, 20 bp, Artificial Sequence
  Antisense Oligonucleotide
atcttctgatattctattag

SEQ ID NO: 121 - DNA, 20 bp, Artificial Sequence
  Antisense Oligonucleotide
aaatgcagtgagtgccatct

SEQ ID NO: 122 - DNA, 20 bp, Artificial Sequence
  Antisense Oligonucleotide
gcacagctccttgaaaggcc

SEQ ID NO: 123 - DNA, 20 bp, Artificial Sequence
  Antisense Oligonucleotide
tgagccatccaccatcgctt

SEQ ID NO: 124 - DNA, 20 bp, Artificial Sequence
  Antisense Oligonucleotide
catgagccatccaccatcgc

SEQ ID NO: 125 - DNA, 20 bp, Artificial Sequence
  Antisense Oligonucleotide
aaccagatgggatgtcggtg

SEQ ID NO: 126 - DNA, 20 bp, Artificial Sequence
  Antisense Oligonucleotide
tggcccaaccccgatcatgg

SEQ ID NO: 127 - DNA, 20 bp, Artificial Sequence
  Antisense Oligonucleotide
tcgaaagcaaatgttggcat

SEQ ID NO: 128 - DNA, 20 bp, Artificial Sequence
  Antisense Oligonucleotide
caatacttggtgcttcctcc

SEQ ID NO: 129 - DNA, 20 bp, Artificial Sequence
  Antisense Oligonucleotide
ggttggagacctcgatgctg

SEQ ID NO: 130 - DNA, 20 bp, Artificial Sequence
  Antisense Oligonucleotide
tgcatcctgatccggatcca

SEQ ID NO: 131 - DNA, 20 bp, Artificial Sequence
  Antisense Oligonucleotide
atctatatctcgaactttaa

SEQ ID NO: 132 - DNA, 20 bp, Artificial Sequence
  Antisense Oligonucleotide
tcaatctatatctcgaactt

SEQ ID NO: 133 - DNA, 20 bp, Artificial Sequence
  Antisense Oligonucleotide
ccaaacatccaggaaataca

SEQ ID NO: 134 - DNA, 20 bp, Artificial Sequence
  Antisense Oligonucleotide
tctttcttggcttgttttaa

SEQ ID NO: 135 - DNA, 20 bp, Artificial Sequence
  Antisense Oligonucleotide
ctgagtcgtgtaccgttggg

SEQ ID NO: 136 - DNA, 20 bp, Artificial Sequence
  Antisense Oligonucleotide
cccactggctgtaaatacgc

SEQ ID NO: 137 - DNA, 20 bp, Artificial Sequence
  Antisense Oligonucleotide
cccgtaattgctccaatctg

SEQ ID NO: 138 - DNA, 20 bp, Artificial Sequence
  Antisense Oligonucleotide
caacccctcccatagcccac

SEQ ID NO: 139 - DNA, 20 bp, Artificial Sequence
  Antisense Oligonucleotide
attgcgctagatgacaccct

SEQ ID NO: 140 - DNA, 20 bp, Artificial Sequence
  Antisense Oligonucleotide
ccttcaattgcgctagatga

SEQ ID NO: 141 - DNA, 20 bp, Artificial Sequence
  Antisense Oligonucleotide
caaaagaatttgccccttca

SEQ ID NO: 142 - DNA, 20 bp, Artificial Sequence
  Antisense Oligonucleotide
tctgaaatataactttagat

SEQ ID NO: 143 - DNA, 20 bp, Artificial Sequence
  Antisense Oligonucleotide
cattacatctgaaatataac

SEQ ID NO: 144 - DNA, 20 bp, Artificial Sequence
  Antisense Oligonucleotide
tttgaatcaaatactatagc

SEQ ID NO: 145 - DNA, 20 bp, Artificial Sequence
  Antisense Oligonucleotide
tctgtacatttaaaacatta

SEQ ID NO: 146 - DNA, 20 bp, Artificial Sequence
  Antisense Oligonucleotide
acaaagtgcaccagttaaat

SEQ ID NO: 147 - DNA, 20 bp, Artificial Sequence
  Antisense Oligonucleotide
tccccagggaatttacaaag

SEQ ID NO: 148 - DNA, 20 bp, Artificial Sequence
  Antisense Oligonucleotide
agctgcaagttttccccagg

SEQ ID NO: 149 - DNA, 20 bp, Artificial Sequence
  Antisense Oligonucleotide
cacaggcttgacaagtctga

SEQ ID NO: 150 - DNA, 20 bp, Artificial Sequence
  Antisense Oligonucleotide
acctggtggccaacatcctg

SEQ ID NO: 151 - DNA, 20 bp, Artificial Sequence
  Antisense Oligonucleotide
tctaaatttgaaaggcacct

SEQ ID NO: 152 - DNA, 20 bp, Artificial Sequence
  Antisense Oligonucleotide
agtggcccattatgtatcct

SEQ ID NO: 153 - DNA, 20 bp, Artificial Sequence
  Antisense Oligonucleotide
ggcacttgtggaaaaacacc

SEQ ID NO: 154 - DNA, 20 bp, Artificial Sequence
  Antisense Oligonucleotide
ttttgctgataaaccactaa

SEQ ID NO: 155 - DNA, 20 bp, Artificial Sequence
  Antisense Oligonucleotide
gttcacaaaggtcctggcag

SEQ ID NO: 156 - DNA, 20 bp, Artificial Sequence
  Antisense Oligonucleotide
gcagactccgccacggcccc

SEQ ID NO: 157 - DNA, 20 bp, Artificial Sequence
  Antisense Oligonucleotide
agcagactccgccacggccc

SEQ ID NO: 158 - DNA, 20 bp, Artificial Sequence
  Antisense Oligonucleotide
ccgccgagcagactccgcca

SEQ ID NO: 159 - DNA, 20 bp, Artificial Sequence
  Antisense Oligonucleotide
tgctccgcgatcgttctctc

SEQ ID NO: 160 - DNA, 20 bp, Artificial Sequence
  Antisense Oligonucleotide
gccctgctccgcgatcgttc

SEQ ID NO: 161 - DNA, 20 bp, Artificial Sequence
  Antisense Oligonucleotide
ccggcgcatggcgcggcgcc

SEQ ID NO: 162 - DNA, 20 bp, Artificial Sequence
  Antisense Oligonucleotide
gctggcccggcgcatggcgc

SEQ ID NO: 163 - DNA, 20 bp, Artificial Sequence
  Antisense Oligonucleotide
tcggctggcccggcgcatgg

SEQ ID NO: 164 - DNA, 20 bp, Artificial Sequence
  Antisense Oligonucleotide
tctcggctggcccggcgcat

SEQ ID NO: 165 - DNA, 20 bp, Artificial Sequence
  Antisense Oligonucleotide
ccttcgtgtgggacgccggg

SEQ ID NO: 166 - DNA, 20 bp, Artificial Sequence
  Antisense Oligonucleotide
cgggaggcggcgggtggcgg

SEQ ID NO: 167 - DNA, 20 bp, Artificial Sequence
  Antisense Oligonucleotide
acatggagcgggaggcggcg

SEQ ID NO: 168 - DNA, 20 bp, Artificial Sequence
  Antisense Oligonucleotide
aacatggagcgggaggcggc

SEQ ID NO: 169 - DNA, 20 bp, Artificial Sequence
  Antisense Oligonucleotide
gctcgaaagtacaggaacag

SEQ ID NO: 170 - DNA, 20 bp, Artificial Sequence
  Antisense Oligonucleotide
tcttcactctccagagtcga

SEQ ID NO: 171 - DNA, 20 bp, Artificial Sequence
  Antisense Oligonucleotide
cagttccttctgcacggccc

SEQ ID NO: 172 - DNA, 20 bp, Artificial Sequence
  Antisense Oligonucleotide
ggcttgcctcgctgggccac

SEQ ID NO: 173 - DNA, 20 bp, Artificial Sequence
  Antisense Oligonucleotide
agcaaatgttggcgtacagg

SEQ ID NO: 174 - DNA, 20 bp, Artificial Sequence
  Antisense Oligonucleotide
agctgaagatagtctgtagg

SEQ ID NO: 175 - DNA, 20 bp, Artificial Sequence
  Antisense Oligonucleotide
ggattttgatgctggtttta

SEQ ID NO: 176 - DNA, 20 bp, Artificial Sequence
  Antisense Oligonucleotide
tcagaattgcccgaccagtt

SEQ ID NO: 177 - DNA, 20 bp, Artificial Sequence
  Antisense Oligonucleotide
atgctaatttcttcaccagc

SEQ ID NO: 178 - DNA, 20 bp, Artificial Sequence
  Antisense Oligonucleotide
tccggatccagcagggaagg

SEQ ID NO: 179 - DNA, 20 bp, Artificial Sequence
  Antisense Oligonucleotide
atgttccacgaaatgagtct

SEQ ID NO: 180 - DNA, 20 bp, Artificial Sequence
  Antisense Oligonucleotide
gtcttacacatgtatagaca

SEQ ID NO: 181 - DNA, 20 bp, Artificial Sequence
  Antisense Oligonucleotide
tcatacaccgtgggccatgt

SEQ ID NO: 182 - DNA, 20 bp, Artificial Sequence
  Antisense Oligonucleotide
accgttgtgtaatcaccatg

SEQ ID NO: 183 - DNA, 20 bp, Artificial Sequence
  Antisense Oligonucleotide
gcatcggaatacctctccca

SEQ ID NO: 184 - DNA, 20 bp, Artificial Sequence
  Antisense Oligonucleotide
tcagtggcacatgtccaggg

SEQ ID NO: 185 - DNA, 20 bp, Artificial Sequence
  Antisense Oligonucleotide
tgcaggtcccagcgcaatgt

SEQ ID NO: 186 - DNA, 20 bp, Artificial Sequence
  Antisense Oligonucleotide
cttatttgcaggtcccagcg

SEQ ID NO: 187 - DNA, 20 bp, Artificial Sequence
  Antisense Oligonucleotide
cattacacctgaaatataac

SEQ ID NO: 188 - DNA, 20 bp, Artificial Sequence
  Antisense Oligonucleotide
aatcaaatactatagcacaa

SEQ ID NO: 189 - DNA, 20 bp, Artificial Sequence
  Antisense Oligonucleotide
taaatattttgaatcaaata

SEQ ID NO: 190 - DNA, 20 bp, Artificial Sequence
  Antisense Oligonucleotide
tgtcaacagtgagacatttt

SEQ ID NO: 191 - DNA, 20 bp, Artificial Sequence
  Antisense Oligonucleotide
gtacatttaaaacattaaat

SEQ ID NO: 192 - DNA, 20 bp, Artificial Sequence
  Antisense Oligonucleotide
tacaaagtgcaccagttaaa

SEQ ID NO: 193 - DNA, 20 bp, Artificial Sequence
  Antisense Oligonucleotide
agctacgagtaccttcaggg

SEQ ID NO: 194 - DNA, 20 bp, Artificial Sequence
  Antisense Oligonucleotide
tttattttgcttgcatagtt

SEQ ID NO: 195 - DNA, 20 bp, Artificial Sequence
  Antisense Oligonucleotide
aaggcacctggtgaccaaca

SEQ ID NO: 196 - DNA, 20 bp, Artificial Sequence
  Antisense Oligonucleotide
tgaaaggcacctggtgacca

SEQ ID NO: 197 - DNA, 20 bp, Artificial Sequence
  Antisense Oligonucleotide
cctgacagatttcagtagcc

SEQ ID NO: 198 - DNA, 20 bp, Artificial Sequence
  Antisense Oligonucleotide
ggaaaaaaaagaaaccaaaa

SEQ ID NO: 199 - DNA, 20 bp, Artificial Sequence
  Antisense Oligonucleotide
cagagacaatgcttttattg

SEQ ID NO: 200 - DNA, 809 bp, R. norvegicus
  CDS (1)...(164)
ggtcgggcaattctgaattccacttttattccataaacgttggaggatttttcaagctccgggctggtgaggaaattagcgtccaggtgtccaacccttccctgttggatccggatcaagatgcgacgtactttggggctttcaaagttcaagacatggactgagacttgttttgtggagccttagcatggatatgctagatgtttggaaggttcttaaaacatggatgatgtagatacatgtgtaagactactaagagacgtggcccacgttgtacaaaactcacggtcctctctcgaccctgtacaggtcatgtatatacaaagtccataggggatattagactcatggtgatcacacaatggtttttacaattttgaaatgaattcctagaattaaaccaaattggaagaggtatacccatgcttataaaaaaaatcgcatgtgagctatggaagggtcatacatagtctctgggtctaactcctgggcctgtgccactgagaacctcgaaattaagagggtaccgttacattgcagagaaatgatggtttgaaaaggtaagttcttttgaattgttacattgcgctggaacctgcaaataagttcttttttctaatgaggagagaaaaatatatgaatttttatataatatctaaagttatatttcaggtgtaaggttttctgtgcaaaatattgtaaattttatttgggctatagtatttgatcaaaaatatttaaaaatgtctcactgttgacatatttaatgttttaaatgtcaagatgtatttaactggtgctctttgtaa

SEQ ID NO: 201 - DNA, 20 bp, Artificial Sequence
  Antisense Oligonucleotide
ggcagcattgatggtgaggt

SEQ ID NO: 202 - DNA, 20 bp, Artificial Sequence
  Antisense Oligonucleotide
ggtacttgccgtagtctcgg

SEQ ID NO: 203 - DNA, 20 bp, Artificial Sequence
  Antisense Oligonucleotide
gcgcaggtacttgccgtagt

SEQ ID NO: 204 - DNA, 20 bp, Artificial Sequence
  Antisense Oligonucleotide
gcgatgctgcagaccacctg

SEQ ID NO: 205 - DNA, 20 bp, Artificial Sequence
  Antisense Oligonucleotide
acagagcgatgctgcagacc

SEQ ID NO: 206 - DNA, 20 bp, Artificial Sequence
  Antisense Oligonucleotide
caggaacagagcgatgctgc

SEQ ID NO: 207 - DNA, 20 bp, Artificial Sequence
  Antisense Oligonucleotide
aagtacaggaacagagcgat

SEQ ID NO: 208 - DNA, 20 bp, Artificial Sequence
  Antisense Oligonucleotide
ctcgaaagtacaggaacaga

SEQ ID NO: 209 - DNA, 20 bp, Artificial Sequence
  Antisense Oligonucleotide
ctgcgctcgaaagtacagga

SEQ ID NO: 210 - DNA, 20 bp, Artificial Sequence
  Antisense Oligonucleotide
tccatctgcgctcgaaagta

SEQ ID NO: 211 - DNA, 20 bp, Artificial Sequence
  Antisense Oligonucleotide
taggatccatctgcgctcga

SEQ ID NO: 212 - DNA, 20 bp, Artificial Sequence
  Antisense Oligonucleotide
tctgttaggatccatctgcg

SEQ ID NO: 213 - DNA, 20 bp, Artificial Sequence
  Antisense Oligonucleotide
gatattctgttaggatccat

SEQ ID NO: 214 - DNA, 20 bp, Artificial Sequence
  Antisense Oligonucleotide
cttctgatattctgttagga

SEQ ID NO: 215 - DNA, 20 bp, Artificial Sequence
  Antisense Oligonucleotide
cgcgtgctgtcttctgatat

SEQ ID NO: 216 - DNA, 20 bp, Artificial Sequence
  Antisense Oligonucleotide
ttctgtagaagcagcgcgtg

SEQ ID NO: 217 - DNA, 20 bp, Artificial Sequence
  Antisense Oligonucleotide
tcacggagtctcagaattct

SEQ ID NO: 218 - DNA, 20 bp, Artificial Sequence
  Antisense Oligonucleotide
aacctgtattttcacggagt

SEQ ID NO: 219 - DNA, 20 bp, Artificial Sequence
  Antisense Oligonucleotide
agtcgagtcctgcaaacctg

SEQ ID NO: 220 - DNA, 20 bp, Artificial Sequence
  Antisense Oligonucleotide
tccagagtcgagtcctgcaa

SEQ ID NO: 221 - DNA, 20 bp, Artificial Sequence
  Antisense Oligonucleotide
tttgcacggccccttgaaag

SEQ ID NO: 222 - DNA, 20 bp, Artificial Sequence
  Antisense Oligonucleotide
tgtaattccctttgcacggc

SEQ ID NO: 223 - DNA, 20 bp, Artificial Sequence
  Antisense Oligonucleotide
tgtggccccacaatgtgttg

SEQ ID NO: 224 - DNA, 20 bp, Artificial Sequence
  Antisense Oligonucleotide
caaacggctgagcctcaggc

SEQ ID NO: 225 - DNA, 20 bp, Artificial Sequence
  Antisense Oligonucleotide
gcagcattgatggtgaggtg

SEQ ID NO: 226 - DNA, 20 bp, Artificial Sequence
  Antisense Oligonucleotide
actttatgggaacccgatgg

SEQ ID NO: 227 - DNA, 20 bp, Artificial Sequence
  Antisense Oligonucleotide
agaggacagactgactttat

SEQ ID NO: 228 - DNA, 20 bp, Artificial Sequence
  Antisense Oligonucleotide
tggtaccaagaggacagact

SEQ ID NO: 229 - DNA, 20 bp, Artificial Sequence
  Antisense Oligonucleotide
gatcatggtaccaagaggac

SEQ ID NO: 230 - DNA, 20 bp, Artificial Sequence
  Antisense Oligonucleotide
atcttggcccagcctcgatc

SEQ ID NO: 231 - DNA, 20 bp, Artificial Sequence
  Antisense Oligonucleotide
tagagatcttggcccagcct

SEQ ID NO: 232 - DNA, 20 bp, Artificial Sequence
  Antisense Oligonucleotide
catgttagagatcttggccc

SEQ ID NO: 233 - DNA, 20 bp, Artificial Sequence
  Antisense Oligonucleotide
agttttccgttgcttaacgt

SEQ ID NO: 234 - DNA, 20 bp, Artificial Sequence
  Antisense Oligonucleotide
acccttagttttccgttgct

SEQ ID NO: 235 - DNA, 20 bp, Artificial Sequence
  Antisense Oligonucleotide
ggttaacccttagttttccg

SEQ ID NO: 236 - DNA, 20 bp, Artificial Sequence
  Antisense Oligonucleotide
atcttggttaacccttagtt

SEQ ID NO: 237 - DNA, 20 bp, Artificial Sequence
  Antisense Oligonucleotide
aagccatcttggttaaccct

SEQ ID NO: 238 - DNA, 20 bp, Artificial Sequence
  Antisense Oligonucleotide
aatagaagccatcttggtta

SEQ ID NO: 239 - DNA, 20 bp, Artificial Sequence
  Antisense Oligonucleotide
taatagaagccatcttggtt

SEQ ID NO: 240 - DNA, 20 bp, Artificial Sequence
  Antisense Oligonucleotide
caggtaatagaagccatctt

SEQ ID NO: 241 - DNA, 20 bp, Artificial Sequence
  Antisense Oligonucleotide
gcgtacaggtaatagaagcc

SEQ ID NO: 242 - DNA, 20 bp, Artificial Sequence
  Antisense Oligonucleotide
tgttggcgtacaggtaatag

SEQ ID NO: 243 - DNA, 20 bp, Artificial Sequence
  Antisense Oligonucleotide
gcaaatgttggcgtacaggt

SEQ ID NO: 244 - DNA, 20 bp, Artificial Sequence
  Antisense Oligonucleotide
agatagtccgcaggtacgct

SEQ ID NO: 245 - DNA, 20 bp, Artificial Sequence
  Antisense Oligonucleotide
accatcagctgaagatagtc

SEQ ID NO: 246 - DNA, 20 bp, Artificial Sequence
  Antisense Oligonucleotide
ttttaacgacatataccatc

SEQ ID NO: 247 - DNA, 20 bp, Artificial Sequence
  Antisense Oligonucleotide
atgctggttttaacgacata

SEQ ID NO: 248 - DNA, 20 bp, Artificial Sequence
  Antisense Oligonucleotide
cttgggattttgatgctggt

SEQ ID NO: 249 - DNA, 20 bp, Artificial Sequence
  Antisense Oligonucleotide
ccagttcttagtgctccccc

SEQ ID NO: 250 - DNA, 20 bp, Artificial Sequence
  Antisense Oligonucleotide
aattcagaattccctgacca

SEQ ID NO: 251 - DNA, 20 bp, Artificial Sequence
  Antisense Oligonucleotide
taaaagtggaattcagaatt

SEQ ID NO: 252 - DNA, 20 bp, Artificial Sequence
  Antisense Oligonucleotide
tggaataaaagtggaattca

SEQ ID NO: 253 - DNA, 20 bp, Artificial Sequence
  Antisense Oligonucleotide
cgcatcttgatccggatcca

SEQ ID NO: 254 - DNA, 20 bp, Artificial Sequence
  Antisense Oligonucleotide
agtacgtcgcatcttgatcc

SEQ ID NO: 255 - DNA, 20 bp, Artificial Sequence
  Antisense Oligonucleotide
cccaaagtacgtcgcatctt

SEQ ID NO: 256 - DNA, 20 bp, Artificial Sequence
  Antisense Oligonucleotide
aaagccccaaagtacgtcgc

SEQ ID NO: 257 - DNA, 20 bp, Artificial Sequence
  Antisense Oligonucleotide
ctttgaaagccccaaagtac

SEQ ID NO: 258 - DNA, 20 bp, Artificial Sequence
  Antisense Oligonucleotide
tcagtctatgtcttgaactt

SEQ ID NO: 259 - DNA, 20 bp, Artificial Sequence
  Antisense Oligonucleotide
gcatatccatgctaaggctc

SEQ ID NO: 260 - DNA, 20 bp, Artificial Sequence
  Antisense Oligonucleotide
gtcttacacatgtatctaca

SEQ ID NO: 261 - DNA, 20 bp, Artificial Sequence
  Antisense Oligonucleotide
cttagtagtcttacacatgt

SEQ ID NO: 262 - DNA, 20 bp, Artificial Sequence
  Antisense Oligonucleotide
ttttgtacaacgtgggccac

SEQ ID NO: 263 - DNA, 20 bp, Artificial Sequence
  Antisense Oligonucleotide
gacctgtacagggtcgagag

SEQ ID NO: 264 - DNA, 20 bp, Artificial Sequence
  Antisense Oligonucleotide
ccattgtgtgatcaccatga

SEQ ID NO: 265 - DNA, 20 bp, Artificial Sequence
  Antisense Oligonucleotide
tagacccagagactatgtat

SEQ ID NO: 266 - DNA, 20 bp, Artificial Sequence
  Antisense Oligonucleotide
gtggcacaggcccaggagtt

SEQ ID NO: 267 - DNA, 20 bp, Artificial Sequence
  Antisense Oligonucleotide
aggttctcagtggcacaggc

SEQ ID NO: 268 - DNA, 20 bp, Artificial Sequence
  Antisense Oligonucleotide
ctctgcaatgtaacggtacc

SEQ ID NO: 269 - DNA, 20 bp, Artificial Sequence
  Antisense Oligonucleotide
aaaccatcatttctctgcaa

SEQ ID NO: 270 - DNA, 20 bp, Artificial Sequence
  Antisense Oligonucleotide
acttatttgcaggttccagc

SEQ ID NO: 271 - DNA, 20 bp, Artificial Sequence
  Antisense Oligonucleotide
gaaaaccttacacctgaaat

SEQ ID NO: 272 - DNA, 20 bp, Artificial Sequence
  Antisense Oligonucleotide
ttttgcacagaaaaccttac

SEQ ID NO: 273 - DNA, 20 bp, Artificial Sequence
  Antisense Oligonucleotide
ccagttaaatacatcttgac

SEQ ID NO: 274 - DNA, 20 bp, M. musculus
ctgccaggacctttgtgaac

SEQ ID NO: 275 - DNA, 20 bp, M. musculus
gggccgtggcggagtctgct

SEQ ID NO: 276 - DNA, 20 bp, M. musculus
tggcggagtctgctcggcgg

SEQ ID NO: 277 - DNA, 20 bp, M. musculus
gagagaacgatcgcggagca

SEQ ID NO: 278 - DNA, 20 bp, M. musculus
gaacgatcgcggagcagggc

SEQ ID NO: 279 - DNA, 20 bp, M. musculus
gcgccatgcgccgggccagc

SEQ ID NO: 280 - DNA, 20 bp, M. musculus
ccatgcgccgggccagccga

SEQ ID NO: 281 - DNA, 20 bp, M. musculus
atgcgccgggccagccgaga

SEQ ID NO: 282 - DNA, 20 bp, M. musculus
ccgccacccgccgcctcccg

SEQ ID NO: 283 - DNA, 20 bp, M. musculus
cgccgcctcccgctccatgt

SEQ ID NO: 284 - DNA, 20 bp, M. musculus
gccgcctcccgctccatgtt

SEQ ID NO: 285 - DNA, 20 bp, M. musculus
ctgttcctgtactttcgagc

SEQ ID NO: 286 - DNA, 20 bp, M. musculus
tcgactctggagagtgaaga

SEQ ID NO: 287 - DNA, 20 bp, M. musculus
gggccgtgcagaaggaactg

SEQ ID NO: 288 - DNA, 20 bp, M. musculus
gtggcccagcgaggcaagcc

SEQ ID NO: 289 - DNA, 20 bp, M. musculus
cctgtacgccaacatttgct

SEQ ID NO: 290 - DNA, 20 bp, M. musculus
cctacagactatcttcagct

SEQ ID NO: 291 - DNA, 20 bp, M. musculus
taaaaccagcatcaaaatcc

SEQ ID NO: 292 - DNA, 20 bp, M. musculus
aactggtcgggcaattctga

SEQ ID NO: 293 - DNA, 20 bp, M. musculus
gctggtgaagaaattagcat

SEQ ID NO: 294 - DNA, 20 bp, M. musculus
ccttccctgctggatccgga

SEQ ID NO: 295 - DNA, 20 bp, M. musculus
agactcatttcgtggaacat

SEQ ID NO: 296 - DNA, 20 bp, M. musculus
tgtctatacatgtgtaagac

SEQ ID NO: 297 - DNA, 20 bp, M. musculus
tgggagaggtattccgatgc

SEQ ID NO: 298 - DNA, 20 bp, M. musculus
ccctggacatgtgccactga

SEQ ID NO: 299 - DNA, 20 bp, M. musculus
acattgcgctgggacctgca

SEQ ID NO: 300 - DNA, 20 bp, M. musculus
cgctgggacctgcaaataag

SEQ ID NO: 301 - DNA, 20 bp, M. musculus
ccctgaaggtactcgtagct

SEQ ID NO: 302 - DNA, 20 bp, M. musculus
aactatgcaagcaaaataaa

SEQ ID NO: 303 - DNA, 20 bp, M. musculus
ggctactgaaatctgtcagg

SEQ ID NO: 304 - DNA, 20 bp, R. norvegicus
ccgagactacggcaagtacc

SEQ ID NO: 305 - DNA, 20 bp, R. norvegicus
actacggcaagtacctgcgc

SEQ ID NO: 306 - DNA, 20 bp, R. norvegicus
caggtggtctgcagcatcgc

SEQ ID NO: 307 - DNA, 20 bp, R. norvegicus
ggtctgcagcatcgctctgt

SEQ ID NO: 308 - DNA, 20 bp, R. norvegicus
gcagcatcgctctgttcctg

SEQ ID NO: 309 - DNA, 20 bp, R. norvegicus
tcctgtactttcgagcgcag

SEQ ID NO: 310 - DNA, 20 bp, R. norvegicus
tcgagcgcagatggatccta

SEQ ID NO: 311 - DNA, 20 bp, R. norvegicus
cgcagatggatcctaacaga

SEQ ID NO: 312 - DNA, 20 bp, R. norvegicus
atggatcctaacagaatatc

SEQ ID NO: 313 - DNA, 20 bp, R. norvegicus
atatcagaagacagcacgcg

SEQ ID NO: 314 - DNA, 20 bp, R. norvegicus
agaattctgagactccgtga

SEQ ID NO: 315 - DNA, 20 bp, R. norvegicus
actccgtgaaaatacaggtt

SEQ ID NO: 316 - DNA, 20 bp, R. norvegicus
caggtttgcaggactcgact

SEQ ID NO: 317 - DNA, 20 bp, R. norvegicus
ttgcaggactcgactctgga

SEQ ID NO: 318 - DNA, 20 bp, R. norvegicus
ctttcaaggggccgtgcaaa

SEQ ID NO: 319 - DNA, 20 bp, R. norvegicus
caacacattgtggggccaca

SEQ ID NO: 320 - DNA, 20 bp, R. norvegicus
ataaagtcagtctgtcctct

SEQ ID NO: 321 - DNA, 20 bp, R. norvegicus
gatcgaggctgggccaagat

SEQ ID NO: 322 - DNA, 20 bp, R. norvegicus
aggctgggccaagatctcta

SEQ ID NO: 323 - DNA, 20 bp, R. norvegicus
acgttaagcaacggaaaact

SEQ ID NO: 324 - DNA, 20 bp, R. norvegicus
agcaacggaaaactaagggt

SEQ ID NO: 325 - DNA, 20 bp, R. norvegicus
cggaaaactaagggttaacc

SEQ ID NO: 326 - DNA, 20 bp, R. norvegicus
aactaagggttaaccaagat

SEQ ID NO: 327 - DNA, 20 bp, R. norvegicus
ggcttctattacctgtacgc

SEQ ID NO: 328 - DNA, 20 bp, R. norvegicus
ctattacctgtacgccaaca

SEQ ID NO: 329 - DNA, 20 bp, R. norvegicus
acctgtacgccaacatttgc

SEQ ID NO: 330 - DNA, 20 bp, R. norvegicus
gactatcttcagctgatggt

SEQ ID NO: 331 - DNA, 20 bp, R. norvegicus
gatggtatatgtcgttaaaa

SEQ ID NO: 332 - DNA, 20 bp, R. norvegicus
accagcatcaaaatcccaag

SEQ ID NO: 333 - DNA, 20 bp, R. norvegicus
aagatgcgacgtactttggg

SEQ ID NO: 334 - DNA, 20 bp, R. norvegicus
gcgacgtactttggggcttt

SEQ ID NO: 335 - DNA, 20 bp, R. norvegicus
gtactttggggctttcaaag

SEQ ID NO: 336 - DNA, 20 bp, R. norvegicus
aagttcaagacatagactga

SEQ ID NO: 337 - DNA, 20 bp, R. norvegicus
gagccttagcatggatatgc

SEQ ID NO: 338 - DNA, 20 bp, R. norvegicus
tgtagatacatgtgtaagac

SEQ ID NO: 339 - DNA, 20 bp, R. norvegicus
acatgtgtaagactactaag

SEQ ID NO: 340 - DNA, 20 bp, R. norvegicus
gtggcccacgttgtacaaaa

SEQ ID NO: 341 - DNA, 20 bp, R. norvegicus
ctctcgaccctgtacaggtc

SEQ ID NO: 342 - DNA, 20 bp, R. norvegicus
tcatggtgatcacacaatgg

SEQ ID NO: 343 - DNA, 20 bp, R. norvegicus
aactcctgggcctgtgccac

SEQ ID NO: 344 - DNA, 20 bp, R. norvegicus
gcctgtgccactgagaacct

SEQ ID NO: 345 - DNA, 20 bp, R. norvegicus
ggtaccgttacattgcagag

SEQ ID NO: 346 - DNA, 20 bp, R. norvegicus
ttgcagagaaatgatggttt

SEQ ID NO: 347 - DNA, 20 bp, R. norvegicus
gctggaacctgcaaataagt

SEQ ID NO: 348 - DNA, 20 bp, Artificial Sequence
  Antisense Oligonucleotide
tgctcagcgagtgtgccagc

SEQ ID NO: 349 - DNA, 25 bp, Artificial Sequence
  primer
tttattccataaatgttgggggatt

SEQ ID NO: 350 - DNA, 22 bp, Artificial Sequence
  primer
ttggacacctgaatgctaattt

SEQ ID NO: 351 - DNA, 23 bp, Artificial Sequence
  probe
ttcaagctccgagctggtgaaga

SEQ ID NO: 352 - DNA, 18 bp, Artificial Sequence
  primer
caacccttccctgctgga

SEQ ID NO: 353 - DNA, 26 bp, Artificial Sequence
  primer
cagtctatgtcctgaactttgaaagc

SEQ ID NO: 354 - DNA, 26 bp, Artificial Sequence
  probe
ccggatcaagatgcgacgtactttgg

SEQ ID NO: 355 - DNA, 20 bp, Artificial Sequence
  antisense oligonucleotide
aggtgctcaggactccattt

SEQ ID NO: 356 - DNA, 20 bp, Artificial Sequence
  antisense oligonucleotide
tcctcgagaggtctccttgc

SEQ ID NO: 357 - DNA, 20 bp, Artificial Sequence
  antisense oligonucleotide
gagctcccggaccctgaggc

SEQ ID NO: 358 - DNA, 20 bp, Artificial Sequence
  antisense oligonucleotide
tcctcgagaggtctccttgc

(19) United States
(12) Patent Application Publication
(10) Pub. No.: US 2005/0060764 A1
(43) Pub. Date: Mar. 17, 2005

(54) Mouse model for bone metabolism

(76) Inventor: Susan Gregory, San Diego, CA (US)

Correspondence Address: FENWICK & WEST LLP, 801 CALIFORNIA STREET, MOUNTAIN VIEW, CA, 94014, US

(21) Appl. No.: 10/667,236

(22) Filed: Sep. 17, 2003

(51) Int. Cl. A01K 67/027

(52) U.S. Cl. 800/18

(57) ABSTRACT

A mouse model for short-term bone metabolism is provided. The mouse is administered parathyroid hormone (PTH), PTH fragments, PTH analogues, parathyroid hormone-related protein (PTHrP), PTHrP fragments, or PTRrP analogues. The mouse model can be used to screen for therapeutic agents for modulating bone metabolism.

### FIELD OF THE INVENTION

The present invention provides a non-invasive animal model of sort-term bone resorption. In particular, this invention is useful for the identification and evaluation of antiresorptive agents, particularly antisense oligonucleotides, in protecting against bone degradation.

### BACKGROUND OF THE INVENTION

Morphogenesis and remodeling of bone are accomplished by the coordinated actions of bone-resorbing osteoclasts and bone-forming osteoblasts, which metabolize and remodel bone structure throughout development and adult life. Bone is constantly being resorbed and formed at specific sites in the skeleton called basic multicellular units. An estimated 10% of the total bone mass in the human body is remodeled each year. Upon activation, osteoclasts, which differentiate from hematopoietic monocyte/macrophage precursors, migrate to the basic multicellular unit, resorb a portion of bone and finally undergo apoptosis. Subsequently, newly generated osteoblasts, arising from preosteoblastic/stromal cells, form bone at the site of resorption. The development of osteoclasts is controlled by preosteoblastic cells, so that the processes of bone resorption and formation are tightly coordinated, thus allowing for a wave of bone formation to follow each cycle of bone resorption. Imbalances between osteoclast and osteoblast activities can result in skeletal abnormalities characterized by decreased (osteoporosis) or increased (osteopetrosis) bone mass (Khosla, Endocrinology, 2001, 142, 5050-5055; Nakashima et al., Curr. Opin. Rheumatol., 2003, 15, 280-287).

Communication between osteoblasts and osteoclasts occurs through cytokines and cell-to-cell contacts. A cytokine that performs a key regulatory role in bone remodeling is receptor activator of NF-kappaB ligand (RANKL). RANKL was first identified as a tumor necrosis factor (TNF) superfamily member [also known as tumor necrosis-factor-related activation-induced cytokine (TRANCE), osteoprotegerin ligand (OPGL), and osteoclast differentiation factor (ODF) and tumor necrosis factor (ligand) superfamily member 11 (TNFSF11)] and was subsequently identified as a factor which is capable of inducing osteoclast differentiation in vitro (Anderson et al., Nature, 1997, 390, 175-179; Lacey et al., Cell, 1998, 93, 165-176; Wong et al., J. Exp. Med., 1997, 186, 2075-2080; Yasuda et al., Proc. Natl. Acad. Sci. U S A, 1998, 95, 3597-3602). The human RANKL gene maps to chromosome 13q14. The highest expression levels of RANKL are found in bone, bone marrow and lymphoid tissues (Anderson et al., Nature, 1997, 390, 175-179; Lacey et al., Cell, 1998, 93, 165-176; Wong et al., J. Exp. Med., 1997, 186, 2075-2080; Yasuda et al., Proc. Natl. Acad. Sci. U S A, 1998, 95, 3597-3602) and it can also be detected in brain, heart, kidney, skeletal muscle and skin (Kartsogiannis et al., Bone, 1999, 25, 525-534).

RANKL is assembled from three RANKL subunits to form the functional trimeric molecule. RANKL is initially anchored to the cell membrane, and a small fraction of the protein released from the cell surface by the proteolytic action of the metalloprotease-disintegrin TNF-alpha convertase (TACE) (Lum et al., J. Biol. Chem., 1999, 274, 13613-13618). RANKL is both necessary and sufficient to stimulate of osteoclast differentiation and activity as well as to inhibit osteoclast apoptosis (Fuller et al., J. Exp. Med., 1998, 188, 997-1001; Lacey et al., Cell, 1998, 93, 165-176; Lum et al., J. Biol. Chem., 1999, 274, 13613-13618; Yasuda et al., Proc. Natl. Acad. Sci. U S A, 1998, 95, 3597-3602).

RANKL is expressed on the surface of preosteoblastic and bone marrow stromal cells. Its expression can be positively or negatively modulated by various hormones, cytokines, growth factors and glucocorticoids, including, vitamin-D3, parathyroid hormone (PTH), interleukin 1-beta and TNF-alpha, all of which increase RANKL expression (Kong et al., Immunol. Today, 2000, 21, 495-502).

At the initiation of the cycle of bone resorption and formation, RANKL binds to its functional receptor RANK on preosteoclastic cells (Anderson et al., Nature, 1997, 390, 175-179; Lacey et al., Cell, 1998, 93, 165-176). This interaction between RANKL and RANK stimulates the formation of mature osteoclasts, which are phenotypically characterized by multinucleation, bone-resorbing function and expression of the lineage specific marker tartrate-resistant acid phosphatase (TRAP) (Burgess et al., J. Cell. Biol., 1999, 145, 527-538; Hsu et al., Proc. Natl. Acad. Sci. U. S. A., 1999, 96, 3540-3545; Lum et al., J. Biol. Chem., 1999, 274, 13613-13618; Yasuda et al., Proc. Natl. Acad. Sci. U S A, 1998, 95, 3597-3602). Alternatively, RANKL can bind to the soluble receptor osteoprotegerin (OPG), which is expressed primarily by bone marrow stromal cells and serves to inhibit osteoclast maturation and activation by RANKL (Lacey et al., Cell, 1998, 93, 165-176; Yasuda et al., Proc. Natl. Acad. Sci. U S A, 1998, 95, 3597-3602). PTH, a major regulator of bone remodeling, stimulates osteoclast function by simultaneously increasing RANKL expression while decreasing OPG expression (Lee and Lorenzo, Endocrinology, 1999, 140, 3552-3561). As preosteoblastic cells differentiate, RANKL mRNA levels are significantly reduced, whereas OPG mRNA levels increase (Gori et al., Endocrinology, 2000, 141, 4768-4776). Such a dynamic relationship between RANKL and OPG levels allows for a wave of osteoclast activity to be followed by a wave osteoblast activity, thereby completing the cycle of bone resorption and formation.

RANKL induces a transient elevation of calcium in osteoclasts due to release of calcium from intracellular stores (Komarova et al., J. Biol. Chem., 2003, 278, 8286-8293). In T-cells, T-cell receptor activation-induced calcium mobilization is solely responsible for the induction of RANKL expression (Wang et al., Eur. J. Immunol., 2002, 32, 1090-1098).

Mice homozygous for disruption of the RANKL gene are born at the expected frequency, but show severely retarded growth after weaning at three weeks of age. RANKL deficient mice exhibit severe osteopetrosis (thickening of bone), defects in tooth eruption and a complete lack of osteoclasts due to the inability of osteoblasts to support osteoclastogenesis (Kong et al., Immunol. Today, 2000, 21, 495-502).

RANKL function is not restricted to bone morphogenesis and remodeling. RANKL-deficient mice also display defects in early differentiation of T- and B-lymphocytes and lack all lymph nodes, demonstrating that RANKL is a regulator of lymph-node organogenesis and lymphocyte development, in addition to being an essential osteoclast differentiation factor (Kong et al., Immunol. Today, 2000, 21, 495-502). T-cell receptor stimulation induces RANKL gene expression, which subsequently leads to activation of c-Jun N-terminal kinase in T-cells (Wong et al., J. Biol. Chem., 1997, 272, 25190-25194). RANKL also participates in immune system function as an important survival factor for bone marrow derived dendritic cells by inhibiting apoptosis in these cells (Lum et al., J. Biol. Chem., 1999, 274, 13613-13618; Wong et al., J. Exp. Med., 1997, 186, 2075-2080). Additionally, RANKL is also required for the development of lobulo-alveolar mammary structures during pregnancy in mice (Fata et al., Cell, 2000, 103, 41-50).

Inappropriate activation of osteoclasts by RANKL can create an imbalance between the processes of bone resorption, resulting in the rate of bone resorption exceeding that of bone formation. Local or generalized bone loss is observed in many osteopenic disorders, including postmenopausal and age-related osteoporosis, periodontitis, familial expansile osteolysis and Paget's disease (Khosla, Endocrinology, 2001, 142, 5050-5055). Upregulation of RANKL mRNA has been reported in several of these diseases.

Paget's disease is characterized by large numbers of abnormal osteoclasts which induce increased bone resorption. RANKL mRNA expression is elevated in both cell lines and bone marrow derived from patients with Paget's disease. Furthermore, osteoclast precursors from Paget's disease patients undergo osteoclastogenesis at a much lower concentration of RANKL than normal cells (Menaa et al., J. Clin. Invest., 2000, 105, 1833-1838).

Other diseases with osteopenic pathologies, such as rheumatoid arthritis, chronic viral infection and adult and child leukemias, are characterized by activated T-cells and bone destruction (Kong et al., Immunol. Today, 2000, 21, 495-502). Rheumatoid arthritis is a chronic inflammatory disease characterized by progressive osteoclast-mediated bone resorption. Rheumatoid arthritis synovial fluid contains osteoclast precursors, RANKL-expressing T-cells and OPG-producing B-cells. Cultured macrophages from rheumatoid arthritis synovial fluid can differentiate into osteoclasts in a RANKL-dependent process (Itonaga et al., J. Pathol., 2000, 192, 97-104). In a T-cell dependent rat model of experimentally-induced arthritis that mimics many of the clinical features of human rheumatoid arthritis, inhibition of RANKL function through OPG treatment prevents bone destruction (Kong et al., Nature, 1999, 402, 304-309).

Multiple myeloma is a cancer in which osteoporosis and bone destruction are prominent features. Myeloma cell lines stimulate RANKL expression while inhibiting OPG expression by bone marrow stromal cells, resulting in a disruption of the balance between RANKL and OPG levels, followed by the aberrant production and activation of osteoclasts (Pearse et al., Proc. Natl. Acad. Sci. U S A, 2001, 98, 11581-11586). A secreted form of RANKL is also expressed by cancer cells responsible for humoral hypercalcemia of malignancy (Nagai et al., Biochem. Biophys. Res. Commun., 2000, 269, 532-536). An increase in RANKL with a concurrent decrease in OPG expression is also observed following glucocorticoid treatment of osteoblastic lineage cells, which also stimulates osteoclastogenesis of these cells, suggesting a mechanism by which systemic glucocorticoid use leads to severe osteoporosis (Hofbauer et al., Endocrinology, 1999, 140, 4382-4389).

These findings demonstrate a link between immune function and bone physiology and also provide a molecular explanation for bone density loss associated with immune disorders and suggest that inhibition of RANKL function, and consequently osteoclast activity, may ameliorate osteopenic conditions (Kong et al., Immunol. Today, 2000, 21, 495-502).

A key mediator of RANKL expression is parathyroid hormone (PTH), a hormone which is synthesized by and secreted from the parathyroid glands in response to changes in extracellular calcium that are detected by a cell-surface calcium-sensing receptor. Increases in serum calcium concentration suppress the release of PTH, whereas decreasing serum calcium stimulates the release of PTH. PTH promotes calcium transport in the gastrointestinal tract and enhances calcium reabsorption in the kidney. Thus, PTH serves an important function as a homeostatic regulator (Rosen and Bilezikian, J. Clin. Endocrinol. Metab., 2001, 86, 957-964; Swarthout et al., Gene, 2002, 282, 1-17).

PTH is also a major regulator of bone remodeling. Continuous administration of PTH to animals stimulates bone resorption by promoting an increase in the actively resorbing osteoclasts. In murine bone marrow cultures, PTH treatment stimulates osteoclast function, which is accompanied by an increase in RANKL expression and a decrease in OPG expression (Lee and Lorenzo, Endocrinology, 1999, 140, 3552-3561). Similarly, continuous infusion of PTH into parathyroidectomized rats causes an increase in RANKL mRNA expression and a decrease in OPG mRNA expression (Ma et al., Endocrinology, 2001, 142, 4047-4054).

Animal models are important to the study of bone metabolism, particularly for the investigation of agents that can relieve osteopenic conditions in humans. PTH replacement in thyroparathyroidectomized rats is a well-established in vivo model of controlled bone resorption (Russell et al., Calcif. Tissue Res., 1970, 6, 183-196). Removal of the thryoid and/or parathyroid eliminates the confounding effects of endogenous parathyroid hormone (Ma et al., Endocrinology, 2001, 142, 4047-4054). Since PTH induces osteoclast-mediated bone resorption, this process is inhibited in parathyroidectomized animals. In addition, because PTH mediates calcium reabsorption for the kidneys and absorption from the small intestine, serum calcium is decreased in parathyroidectomized animals, and the animals remain in a hypocalcemic state. Controlled PTH replacement results in serum calcium increase due to PTH-induced osteoclast-mediated bone degradation, thus, serum calcium can be used to measure bone resorption (Swarthout et al., Gene, 2002, 282, 1-17).

More recent studies of the PTH replacement model involve removal of the parathyroid only (Ma et al., Endocrinology, 2001, 142, 4047-4054). Technically, it is very difficult to remove only the parathyroid gland of rodents. The thyroid glands frequently are inadvertently removed during the surgical procedure, and the resultant decrease in thyroid hormone levels can have undesired effects on bone turnover that can contribute to unacceptable levels of experimental variability in this model. Development of a short-term bone resorption model that does not involve the parathyroidectomy procedure is therefore highly desirable. This model could be used to examine the ability of agents to inhibit bone resorption, which is observed in association with many conditions, including age-related and post-menopausal osteoporosis, glucocorticoid-induced osteoporosis, rheumatoid arthritis and other chronic inflammatory conditions, and hypercalcemia associated with cancer (Kong et al., Immunol. Today, 2000, 21, 495-502). A short-term, non-invasive bone resorption model is useful for the investigation of genes involved in bone resorption for the purpose of understanding bone metabolism, as well as identifying new targets for antiresorptive compounds. This invention describes such a model.

The U.S. Pat. No. 5,997,101 discloses the use of a thyroparathyroidectomized rat model in the study of compounds that effect the calcemic response to parathyroid hormone (Ali et al., 1999).

### SUMMARY OF THE INVENTION

The present invention is directed to a mouse model for bone metabolism disease. The mouse model is a short-term mouse model of bone resorption created by the infusion of parathyroid hormone (PTH), its analogues or its fragments.

In one aspect, the mouse model of the invention comprising a mouse exposed to a compound selected from the group consisting of parathyroid hormone (PTH), an analogue of PTH, and a fragment of PTH for a time sufficient whereby serum calcium concentration and RANKL mRNA expression are increased in the model. The mouse is exposed to about 0.5 ·g to about 8 ·g of the compound per 100 g of bodyweight, and the serum calcium concentration and RANKL mRNA expression are increased by about 10% or higher.

In another aspect, the invention related to a method for testing a mouse model for bone metabolism disease. The method comprising administering to the mouse an antisense oligonucleotide to RANK or RANKL; administering to the mouse a compound selected from the group consisting of parathyroid hormone (PTH), an analogue of PTH, and a fragment of PTH; and assessing the affect of the antisense oligonucleotide on the mouse compared to a control mouse not treated with the antisense oligonucleotide. The antisense oligonucleotide inhibits an increase in RANKL mRNA expression, RANK mRNA expression, and/or an increase in serum calcium concentration, by at least about 10% compared to the control.

### DETAILED DESCRIPTION OF THE INVENTION

A. Overview of the Invention

The present invention employs compounds, preferably oligonucleotides and similar species for use in modulating the function or effect of nucleic acid molecules encoding RANKL. This is accomplished by providing oligonucleotides which specifically hybridize with one or more nucleic acid molecules encoding RANKL. As used herein, the terms “target nucleic acid” and “nucleic acid molecule encoding RANKL” have been used for convenience to encompass DNA encoding RANKL, RNA (including pre-mRNA and mRNA or portions thereof) transcribed from such DNA, and also cDNA derived from such RNA. The hybridization of a compound of this invention with its target nucleic acid is generally referred to as an “antisense compound”. Consequently, the preferred mechanism believed to be included in the practice of some preferred embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently preferred to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which can be engaged in or facilitated by the RNA. One preferred result of such interference with target nucleic acid function is modulation of the expression of RANKL. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the preferred form of modulation of expression and mRNA is often a preferred target nucleic acid.

In the context of this invention, “hybridization” means the pairing of complementary strands of oligomeric compounds. In the present invention, the preferred mechanism of pairing involves hydrogen bonding, which can be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An antisense compound is specifically hybridizable when binding of the compound to the target nucleic acid interferes with the normal function of the target nucleic acid to cause a loss of activity, and there is a sufficient degree of complementarity to avoid non-specific binding of the antisense compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing between two nucleobases of an oligomeric compound. For example, if a nucleobase at a certain position of an oligonucleotide (an oligomeric compound), is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid is considered to be a complementary position. The oligonucleotide and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense compound need not be 100% complementary to that of its target nucleic acid to be specifically hybridizable. Moreover, an oligonucleotide can hybridize over one or more segments such that intervening or adjacent segments are not involved in the hybridization event (e.g., a loop structure or hairpin structure It is preferred that the antisense compounds of the present invention comprise at least 70%, or at least 75%, or at least 80%, or at least 85% sequence complementarity to a target region within the target nucleic acid, more preferably that they comprise at least 90% sequence complementarity and even more preferably comprise at least 95% or at least 99% sequence complementarity to the target region within the target nucleic acid sequence to which they are targeted. For example, an antisense compound in which 18 of 20 nucleobases of the antisense compound are complementary to a target region, and would therefore specifically hybridize, would represent 90 percent complementarity. In this example, the remaining noncomplementary nucleobases can be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some preferred embodiments, homology, sequence identity or complementarity, between the oligomeric and target is between about 50% to about 60%. In some embodiments, homology, sequence identity or complementarity, is between about 60% to about 70%. In preferred embodiments, homology, sequence identity or complementarity, is between about 70% and about 80%. In more preferred embodiments, homology, sequence identity or complementarity, is between about 80% and about 90%. In some preferred embodiments, homology, sequence identity or complementarity, is about 90%, about 92%, about 94%, about 95%, about 96%, about 97%, about 98%, or about 99% .

B. Compounds of the Invention

According to the present invention, compounds include antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these compounds can be introduced in the form of single-stranded, double-stranded, circular or hairpin oligomeric compounds and can contain structural elements such as internal or terminal bulges or loops. Once introduced to a system, the compounds of the invention can elicit the action of one or more enzymes or structural proteins to effect modification of the target nucleic acid. One non-limiting example of such an enzyme is RNAse H, a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While the preferred form of antisense compound is a single-stranded antisense oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing. Accordingly, in one embodiment of the invention, the antisense compound is a double stranded structure, e.g., a dsRNA.

The first evidence that dsRNA could lead to gene silencing in animals came in 1995 from work in the nematode, Caenorhabditis elegans (Guo and Kempheus, Cell, 1995, 81, 611-620). Montgomery et al. have shown that the primary interference effects of dsRNA are posttranscriptional (Montgomery et al., Proc. Natl. Acad. Sci. USA, 1998, 95, 15502-15507). The posttranscriptional antisense mechanism defined in Caenorhabditis elegans resulting from exposure to double-stranded RNA (dsRNA) has since been designated RNA interference (RNAi). This term has been generalized to mean antisense-mediated gene silencing involving the introduction of dsRNA leading to the sequence-specific reduction of endogenous targeted mRNA levels (Fire et al., Nature, 1998, 391, 806-811). Recently, it has been shown that it is, in fact, the single-stranded RNA oligomers of antisense polarity of the dsRNAs which are the potent inducers of RNAi (Tijsterman et al., Science, 2002, 295, 694-697).

The oligonucleotides of the present invention also include modified oligonucleotides in which a different base is present at one or more of the nucleotide positions in the oligonucleotide, as long as the structural and functional elements are maintained, e.g., the modified oligonucleotide hybridizes to and inhibits the expression of the target gene. For example, if the first nucleotide is an adenosine, modified oligonucleotides can be produced which contain thymidine, guanosine or cytidine at this position. This can be done at any of the positions of the oligonucleotide. These oligonucleotides are then tested using the methods described herein to determine their ability to inhibit expression of RANKL mRNA.

In the context of this invention, the term “oligomeric compound” refers to a polymer or oligomer comprising a plurality of monomeric units. In the context of this invention, the term “oligonucleotide” refers to an oligomer or polymer of ribonucleic acid (RNA) or deoxyribonucleic acid (DNA) or mimetics, chimeras, analogs and homologs thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for a target nucleic acid and increased stability in the presence of nucleases.

While oligonucleotides are a preferred form of the compounds of this invention, the present invention comprehends other families of compounds as well, including but not limited to oligonucleotide analogs and mimetics such as those described herein.

The compounds in accordance with this invention preferably comprise from about 8 to about 80 nucleobases (i.e. from about 8 to about 80 linked nucleosides). One of ordinary skill in the art will appreciate that the invention embodies compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In one preferred embodiment, the compounds of the invention are 12 to 50 nucleobases in length. One having ordinary skill in the art will appreciate that this embodies compounds of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In another preferred embodiment, the compounds of the invention are 15 to 30 nucleobases in length. One having ordinary skill in the art will appreciate that this embodies compounds of 15, 16, 17, 18, 19, 20, 21, 22, 23, 24, 25, 26, 27, 28, 29, or 30 nucleobases in length.

Particularly preferred compounds are oligonucleotides from about 12 to about 50 nucleobases, even more preferably those comprising from about 15 to about 30 nucleobases.

Antisense compounds 8-80 nucleobases in length comprising a stretch of at least eight (8) consecutive nucleobases selected from within the illustrative antisense compounds are considered to be suitable antisense compounds as well.

Exemplary preferred antisense compounds include oligonucleotide sequences that comprise at least the 8 consecutive nucleobases from the 5′-terminus of one of the illustrative preferred antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Similarly preferred antisense compounds are represented by oligonucleotide sequences that comprise at least the 8 consecutive nucleobases from the 3′-terminus of one of the illustrative preferred antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). One having skill in the art armed with the preferred antisense compounds illustrated herein will be able, without undue experimentation, to identify further preferred antisense compounds.

C. Targets of the Invention

“Targeting” an antisense compound to a particular nucleic acid molecule, in the context of this invention, can be a multistep process. The process usually begins with the identification of a target nucleic acid whose function is to be modulated. This target nucleic acid can be, for example, a cellular gene (or mRNA transcribed from the gene) whose expression is associated with a particular disorder or disease state, or a nucleic acid molecule from an infectious agent. In the present invention, the target nucleic acid encodes RANKL.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes can have two or more alternative start codons, any one of which can be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding RANKL, regardless of the sequence(s) of such codons. It is also known in the art that a translation termination codon (or “stop codon”) of a gene can have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which can be targeted effectively with the antisense compounds of the present invention.

The open reading frame (ORF) or “coding region,” which is known in the art to refer to the region between the translation initiation codon and the translation termination codon, is also a region which can be targeted effectively. Within the context of the present invention, a preferred region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also preferred to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, can also be particularly useful in situations where aberrant splicing is implicated in disease, or where an overproduction of a particular splice product is implicated in disease. Aberrant fusion junctions due to rearrangements or deletions are also preferred target sites. mRNA transcripts produced via the process of splicing of two (or more) mRNAs from different gene sources are known as “fusion transcripts”. It is also known that introns can be effectively targeted using antisense compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequence.

Upon excision of one or more exon or intron regions, or portions thereof during splicing, pre-mRNA variants produce smaller “mRNA variants”. Consequently, mRNA variants are processed pre-mRNA variants and each unique pre-mRNA variant must always produce a unique mRNA variant as a result of splicing. These mRNA variants are also known as “alternative splice variants”. If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also preferred target nucleic acids.

The locations on the target nucleic acid to which the preferred antisense compounds hybridize are hereinbelow referred to as “preferred target segments.” As used herein the term “preferred target segment” is defined as at least an 8-nucleobase portion of a target region to which an active antisense compound is targeted. While not wishing to be bound by theory, it is presently believed that these target segments represent portions of the target nucleic acid which are accessible for hybridization.

While the specific sequences of certain preferred target segments are set forth herein, one of skill in the art will recognize that these serve to illustrate and describe particular embodiments within the scope of the present invention. Additional preferred target segments can be identified by one having ordinary skill.

Target segments 8-80 nucleobases in length comprising a stretch of at least eight (8) consecutive nucleobases selected from within the illustrative preferred target segments are considered to be suitable for targeting as well.

Target segments can include DNA or RNA sequences that comprise at least the 8 consecutive nucleobases from the 5′-terminus of one of the illustrative preferred target segments (the remaining nucleobases being a consecutive stretch of the same DNA or RNA beginning immediately upstream of the 5′-terminus of the target segment and continuing until the DNA or RNA contains about 8 to about 80 nucleobases). Similarly preferred target segments are represented by DNA or RNA sequences that comprise at least the 8 consecutive nucleobases from the 3′-terminus of one of the illustrative preferred target segments (the remaining nucleobases being a consecutive stretch of the same DNA or RNA beginning immediately downstream of the 3′-terminus of the target segment and continuing until the DNA or RNA contains about 8 to about 80 nucleobases). One having skill in the art armed with the preferred target segments illustrated herein will be able, without undue experimentation, to identify further preferred target segments.

Once one or more target regions, segments or sites have been identified, antisense compounds are chosen which are sufficiently complementary to the target, i.e., hybridize sufficiently well and with sufficient specificity, to give the desired effect.

The oligomeric compounds are also targeted to or not targeted to regions of the target nucleobase sequence (e.g., such as those disclosed in Example 13) comprising nucleobases 1-50, 51-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, 2001-2050, 2051-2100, 2101-2150, 2151-2200, 2201-2250, 2251-2271, or any combination thereof.

D. Screening and Target Validation

In a further embodiment, the “preferred target segments” identified herein can be employed in a screen for additional compounds that modulate the expression of RANKL. “Modulators” are those compounds that decrease or increase the expression of a nucleic acid molecule encoding RANKL and which comprise at least an 8-nucleobase portion which is complementary to a preferred target segment. The screening method comprises the steps of contacting a preferred target segment of a nucleic acid molecule encoding RANKL with one or more candidate modulators, and selecting for one or more candidate modulators which decrease or increase the expression of a nucleic acid molecule encoding RANKL. Once it is shown that the candidate modulator or modulators are capable of modulating (e.g. either decreasing or increasing) the expression of a nucleic acid molecule encoding RANKL, the modulator can then be employed in further investigative studies of the function of RANKL, or for use as a research, diagnostic, or therapeutic agent in accordance with the present invention.

The preferred target segments of the present invention can be also be combined with their respective complementary antisense compounds of the present invention to form stabilized double-stranded (duplexed) oligonucleotides.

Such double stranded oligonucleotide moieties have been shown in the art to modulate target expression and regulate translation as well as RNA processing via an antisense mechanism. Moreover, the double-stranded moieties can be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The compounds of the present invention can also be applied in the areas of drug discovery and target validation. The present invention comprehends the use of the compounds and preferred target segments identified herein in drug discovery efforts to elucidate relationships that exist between RANKL and a disease state, phenotype, or condition. These methods include detecting or modulating RANKL comprising contacting a sample, tissue, cell, or organism with the compounds of the present invention, measuring the nucleic acid or protein level of RANKL and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

E. Kits, Research Reagents, Diagnostics, and Therapeutics

The compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense compounds are compared to control cells or tissues not treated with antisense compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U. S. A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compounds of the invention are useful for research and diagnostics, because these compounds hybridize to nucleic acids encoding RANKL. For example, oligonucleotides that are shown to hybridize with such efficiency and under such conditions as disclosed herein as to be effective RANKL inhibitors will also be effective primers or probes under conditions favoring gene amplification or detection, respectively. These primers and probes are useful in methods requiring the specific detection of nucleic acid molecules encoding RANKL and in the amplification of said nucleic acid molecules for detection or for use in further studies of RANKL. Hybridization of the antisense oligonucleotides, particularly the primers and probes, of the invention with a nucleic acid encoding RANKL can be detected by means known in the art. Such means can include conjugation of an enzyme to the oligonucleotide, radiolabelling of the oligonucleotide or any other suitable detection means. Kits using such detection means for detecting the level of RANKL in a sample can also be prepared.

The specificity and sensitivity of antisense is also harnessed by those of skill in the art for therapeutic uses. Antisense compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense compounds can be useful therapeutic modalities that can be configured to be useful in treatment regimes for the treatment of cells, tissues and animals, especially humans.

For therapeutics, an animal, preferably a human, suspected of having a disease or disorder which can be treated by modulating the expression of RANKL is treated by administering antisense compounds in accordance with this invention. For example, in one non-limiting embodiment, the methods comprise the step of administering to the animal in need of treatment, a therapeutically effective amount of a RANKL inhibitor. The RANKL inhibitors of the present invention effectively inhibit the activity of the RANKL protein or inhibit the expression of the RANKL protein. In one embodiment, the activity or expression of RANKL in an animal is inhibited by about 10%. Preferably, the activity or expression of RANKL in an animal is inhibited by about 30%. More preferably, the activity or expression of RANKL in an animal is inhibited by 50% or more. Thus, the oligomeric compounds modulate expression of RANKL mRNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of RANKL can be measured in serum, adipose tissue, liver or any other body fluid, tissue or organ of the animal. Preferably, the cells contained within said fluids, tissues or organs being analyzed contain a nucleic acid molecule encoding RANKL protein and/or the RANKL protein itself.

The compounds of the invention can be utilized in pharmaceutical compositions by adding an effective amount of a compound to a suitable pharmaceutically acceptable diluent or carrier. Use of the compounds and methods of the invention can also be useful prophylactically.

F. Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally preferred. In addition, linear compounds can have internal nucleobase complementarity and can therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages (Backbones)

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones containing a phosphorus atom therein include, for example, phosphorothioates, chiral phosphorothioates, phosphoro-dithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e. a single inverted nucleoside residue which can be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Modified Sugar and Internucleoside Linkages-Mimetics

In other preferred oligonucleotide mimetics, both the sugar and the internucleoside linkage (i.e. the backbone), of the nucleotide units are replaced with novel groups. The nucleobase units are maintained for hybridization with an appropriate target nucleic acid. One such compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified Sugars

Modified oligonucleotides can also contain one or more substituted sugar moieties. Preferred oligonucleotides comprise one of the following at the 2′ position: OH; F; O—, S—, or N-alkyl; O—, S—, or N-alkenyl; O—, S- or N-alkynyl; or O-alkyl—O—alkyl, wherein the alkyl, alkenyl and alkynyl can be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O—(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O—dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O—allyl (2′-O—CH₂—CH═CH₂) and 2′-fluoro (2′-F). The 2′-modification can be in the arabino (up) position or ribo (down) position. A preferred 2′-arabino modification is 2′-F. Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

A further preferred modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is preferably a methylene (—CH₂-), group bridging the 2′ oxygen atom and the 4′ carbon atom wherein n is 1 or 2. LNAs and preparation thereof are described in WO 98/39352 and WO 99/14226.

Natural and Modified Nucleobases

Oligonucleotides can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′: 4,5]pyrrolo[2,3-d]pyrimidin-2-one). Modified nucleobases can also include those in which the purine or pyrimidine base is replaced with other heterocycles, for example 7-deaza-adenine, 7-deazaguanosine, 2-aminopyridine and 2-pyridone. Further nucleobases include those disclosed in United States Patent No. 3,687,808, those disclosed in The Concise Encyclopedia of Polymer Science and Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Conjugates Another modification of the oligonucleotides of the invention involves chemically linking to the oligonucleotide one or more moieties or conjugates which enhance the activity, cellular distribution or cellular uptake of the oligonucleotide. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. No. 6,287,860, the entire disclosure of which are incorporated herein by reference. Conjugate moieties include but are not limited to lipid moieties such as a cholesterol moiety, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di—O—hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligonucleotides of the invention can also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent Application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Chimeric Compounds

It is not necessary for all positions in a given compound to be uniformly modified, and in fact more than one of the aforementioned modifications can be incorporated in a single compound or even at a single nucleoside within an oligonucleotide.

The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, increased stability and/or increased binding affinity for the target nucleic acid. An additional region of the oligonucleotide can serve as a substrate for enzymes capable of cleaving RNA:DNA or RNA:RNA hybrids. By way of example, RNAse H is a cellular endonuclease which cleaves the RNA strand of an RNA:DNA duplex. Activation of RNase H, therefore, results in cleavage of the RNA target, thereby greatly enhancing the efficiency of oligonucleotide-mediated inhibition of gene expression. The cleavage of RNA:RNA hybrids can, in like fashion, be accomplished through the actions of endoribonucleases, such as RNAseL which cleaves both cellular and viral RNA. Cleavage of the RNA target can be routinely detected by gel electrophoresis and, if necessary, associated nucleic acid hybridization techniques known in the art.

Chimeric antisense compounds of the invention can be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, certain of which are commonly owned with the instant application, and each of which is herein incorporated by reference in its entirety.

G. Formulations

The compounds of the invention can also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligonucleotides, preferred examples of pharmaceutically acceptable salts and their uses are further described in U.S. Pat. No. 6,287,860, which is incorporated herein in its entirety.

The present invention also includes pharmaceutical compositions and formulations which include the antisense compounds of the invention. The pharmaceutical compositions of the present invention can be administered in a number of ways depending upon whether local or systemic treatment is desired and upon the area to be treated. Administration can be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O—methoxyethyl modification are believed to be particularly useful for oral administration. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

Pharmaceutical compositions of the present invention include, but are not limited to, solutions, emulsions, foams and liposome-containing formulations. The pharmaceutical compositions and formulations of the present invention can comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions can contain additional components in addition to the dispersed phases, and the active drug which can be present as a solution in either the aqueous phase, oily phase or itself as a separate phase. Microemulsions are included as an embodiment of the present invention. Emulsions and their uses are well known in the art and are further described in U.S. Patent 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860, which is incorporated herein in its entirety.

The pharmaceutical formulations and compositions of the present invention can also include surfactants. The use of surfactants in drug products, formulations and in emulsions is well known in the art. Surfactants and their uses are further described in U.S. Pat. No. 6,287,860, which is incorporated herein in its entirety.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers can be classified as belonging to one of five broad categories, i.e., surfactants, fatty acids, bile salts, chelating agents, and non-chelating non-surfactants. Penetration enhancers and their uses are further described in U.S. Pat. No. 6,287,860, which is incorporated herein in its entirety.

One of skill in the art will recognize that formulations are routinely designed according to their intended use, i.e. route of administration.

Preferred formulations for topical administration include those in which the oligonucleotides of the invention are in admixture with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention can be encapsulated within liposomes or can form complexes thereto, in particular to cationic liposomes. Alternatively, oligonucleotides can be complexed to lipids, in particular to cationic lipids. Preferred fatty acids-and esters, pharmaceutically acceptable salts thereof, and their. uses are further described in U.S. Pat. No. 6,287,860, which is incorporated herein in its entirety. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. Preferred oral formulations are those in which oligonucleotides of the invention are administered in conjunction with one or more penetration enhancers surfactants and chelators. Preferred surfactants include fatty acids and/or esters or salts thereof, bile acids and/or salts thereof. Preferred bile acids/salts and fatty acids and their uses are further described in U.S. Pat. No. 6,287,860, which is incorporated herein in its entirety. Also preferred are combinations of penetration enhancers, for example, fatty acids/salts in combination with bile acids/salts. A particularly preferred combination is the sodium salt of lauric acid, capric acid and UDCA. Further penetration enhancers include polyoxyethylene-9-lauryl ether, polyoxyethylene-20-cetyl ether. Oligonucleotides of the invention can be delivered orally, in granular form including sprayed dried particles, or complexed to form micro or nanoparticles. Oligonucleotide complexing agents and their uses are further described in U.S. Pat. No. 6,287,860, which is incorporated herein in its entirety. Oral formulations for oligonucleotides and their preparation are described in detail in U.S. applications Ser. No. 09/108,673 (filed Jul. 1, 1998), Ser. No. 09/315,298 (filed May 20, 1999) and Ser. No. 10/071,822, filed Feb. 8, 2002, each of which is incorporated herein by reference in their entirety.

Compositions and formulations for parenteral, intrathecal or intraventricular administration can include sterile aqueous solutions which can also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more oligomeric compounds and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compounds of the invention, such chemotherapeutic agents can be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, can also be combined in compositions of the invention. Combinations of antisense compounds and other non-antisense drugs are also within the scope of this invention. Two or more combined compounds can be used together or sequentially.

In another related embodiment, compositions of the invention can contain one or more antisense compounds, particularly oligonucleotides, targeted to a first nucleic acid and one or more additional antisense compounds targeted to a second nucleic acid target. Alternatively, compositions of the invention can contain two or more antisense compounds targeted to different regions of the same nucleic acid target. Numerous examples of antisense compounds are known in the art. Two or more combined compounds can be used together or sequentially.

H. Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages can vary depending on the relative potency of individual oligonucleotides, and can generally be estimated based on EC₅₀s found to be effective in in vitro and in vivo animal models. In general, dosage is from 0.01 ug to 100 g per kg of body weight, and can be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it can be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its preferred embodiments, the following examples serve only to illustrate the invention and are not intended to limit the same. Each of the references, GenBank accession numbers, and the like recited in the present application are incorporated herein by reference in its entirety.

H. Mouse Model

In one aspect of the invention, animal models are provided. Such models are useful for investigating bone metabolism in organisms, and genes associated with bone metabolism, studying bone density or bone mass modulation and the development of methods and treatments for modulating bone metabolism, affecting bone density or modulation of bone mass. The animal model is preferably a mouse model, but can include any non-human animal, such as, for example, primates, canines, felines, rodents, ovines, bovines, and the like. When the animal model is a mouse, the mouse can be any type of a mouse, such as Swiss-Webster mouse, Black-Swiss mouse, Rhino-mutant mouse, ICR mouse, and the like that are available from commercial sources or custom sources.

The mouse can be administered parathyroid hormone (PTH), PTH fragments, PTH analogues, parathyroid hormone-related protein (PTHrP), PTHrP fragments, or PTRrP analogues. Parathyroid hormone or a biologically active fragment thereof such as is available from Sigma Chemical Company, (St. Louis, Mo., 63103, see catalog and references therein). A preferred parathyroid hormone is parathyroid hormone 1-34, which can be obtained from Sigma Chemical Company. Another preferred parathyroid hormone is parathyroid hormone 1-38, which can be obtained from Sigma Chemical Company. In addition, when the hormonal compounds mentioned above and pharmaceutically acceptable salts thereof used in the compositions and methods of the present invention form hydrates or solvates, such hydrates or solvates are also within the scope of the invention.

Typically, the animal, such as the mouse model, can be exposed to PTH, an analogue of PTH, a fragment of PTH, PTHrP, an analogue of PTHrP, or a fragment of PTHrP. The exposure of the compounds can be via any method which can deliver a compound systemically and/or locally. These methods include oral routes, parenteral routes, intraduodenal routes, transdermal administration, and the like. Generally, the compounds can be administered parenterally, such as by intravenous, intramuscular, transcutaneous, subcutaneous or intramedullary. The animal, such as mouse, can be administered a dose of about 0.1 ug to about 15 ug of the compound, preferably about 0.5 ug to about 8 ug of the compound, or even more preferably about 1 ug to about 5 ug of the compound per 100 g of the bodyweight, or any dose in between. The dose is preferably administered continuously from about 0.5 h to about 96 h, preferably from about 6 h to about 48 h, or more preferably from about 20 h to about 28 h, or any time in between. The animals thus treated can be used as short-term, non-invasive models for investigating bone metabolism.

In one aspect, the mouse models described above are used to assess the effect of an agent on bone metabolism. The agent can be, for example, useful for the treatment and/or prevention of osteoporosis, can increase bone formation by which is meant that the rate of bone formation in a subject administered the agent is increased over the bone formation rate in a subject that is not given the agent, to postpone the development of bone loss symptoms and/or a reduction in the severity of such symptoms that will or are expected to develop, ameliorating existing bone or cartilage deficit symptoms, preventing additional symptoms, ameliorating or preventing the underlying metabolic causes of symptoms, and/or encouraging bone growth. The agent can be any known or new compound, or can be one or more of the antisense oligonucleotides described in detail above.

Briefly, the effect of the test compound is assayed by injecting test amounts of the agent selected into a mouse or other experimental animal, followed by exposure to PTH, an analogue of PTH, a fragment of PTH, PTHrP, an analogue of PTHrP, or a fragment of PTHrP, as described above. When the test compound is an antisense oligonucleotide, it can be administered for about 1 day to about 30 days, preferably for about 5 days to about 20 days, or more preferably for about 12 days to about 18 days, or any time period in between. As one of skill in the art will recognize, the test compound can be administered for longer period of time if desired. The concentration of the antisense oligonucleotide can be from about 1 mg/kg/day to about 200 mg/kg/day, preferably about 5 mg/kg/day to about 100 mg/kg/day, or more preferably about 10 mg/kg/day to about 50 mg/kg/day.

The modulation of bone metabolism by the test compound, such an antisense oligonucleotide described above, can be determined by measuring the serum calcium concentration, the level of RANKL mRNA expression, and/or the level of RANK mRNA expression. The modulation of bone metabolism can be an inhibition or an increase in bone formation as measured by one of these markers. In this context, inhibition or increase refers to a lower level or higher level of measured activity relative to a control experiment in which the enzyme, cell, or subject is not treated with the test compound. In particular aspects, the modulation of bone metabolism is at least a 10% inhibition or increase over control. One of skill in the art will appreciate that modulation of at least 20%, 50%, 75%, 90% or 100% or any integer between 10% and 100%, can be preferred for particular applications.

In another aspect, the modulation of bone metabolism by the test compound can be determine by examination of bone strength and mass after administration compared to a control subject. Such examination can be performed in situ by using imaging techniques (e.g., nuclear magnetic resonance imaging, X-ray tomography, ultrasound, and sound conduction) or stress testing, or ex vivo by standard histological methods. Modulation of bone density and/or bone mass can be assessed by changes in one or more parameters such as bone mineral density, bone strength, trabecular number, bone size, and bone tissue connectivity. Several methods for determining bone mineral density (BMD) are known in the art. For example, BMD measurements can be done using, e.g., dual energy xray absorptiometry or quantitative computed tomography, and the like. Similarly, increased bone formation can be determined using methods well known in the art. For example, dynamic measurements of bone formation rate (BFR) can be performed on tetracycline labeled cancellous bone from the lumbar spine and distal femur metaphysis using quantitative digitized morphometry (see, e.g., Ling et al., Endocrinology (1999) 140:5780-5788. Alternatively, bone formation markers, such as alkaline phosphatase activity and serum osteocalcin levels can be assessed to indirectly determine whether increased bone formation has occurred (see Looker et al., Osteoporosis International (2000) 11(6):467-480). The compounds thus identified as modulators of bone metabolism can be subsequently tested for their ability to prevent bone loss and/or promote bone formation.

### EXAMPLES

### Example 1

Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴_ benzoyl-5-methyl-cytidine penultimate intermediate, [5′-O-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amdite), (5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylamino-oxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(1 2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy)nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy (2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 2

Oligonucleotide and Oligonucleoside Synthesis

The antisense compounds used in accordance with this invention can be conveniently and routinely made through the well-known technique of solid phase synthesis. Equipment for such synthesis is sold by several vendors including, for example, Applied Biosystems (Foster City, Calif.). Any other means for such synthesis known in the art can additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. Nos. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligo-nucleosides, also identified as amide-4 linked oligonucleosides, as well as mixed backbone compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 3

RHA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acidlabile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA antisense compounds (RNA oligonucleotides) of the present invention can be synthesized by the methods herein or purchased from Dharmacon Research, Inc (Lafayette, Colo.). Once synthesized, complementary RNA antisense compounds can then be annealed by methods known in the art to form double stranded (duplexed) antisense compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense compounds can be used in kits, assays, screens, or other methods to investigate the role of a target nucleic acid.

### Example 4

Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap′ segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers, or “wingmers”.

### [2′-O-Me]-[2′-deoxy]-[2′-O-Me] Chimeric Phosphorothioate Oligonuclootides

Chimeric oligonucleotides having 2′-O-alkyl phosphorothioate and 2′-deoxy phosphorothioate oligo-nucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphor-amidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 552° C. The deprotected oligo is then recovered by an 25 appropriate method (precipitation, column chromatography, volume reduced in vacuo and analyzed spectrophotometrically for yield and for purity by capillary electrophoresis and by mass spectrometry.

### [2′-O-(2-Methoxyethyl)]-[2′-deoxy]-[2′-O-(Mothoxyothyl)] Chimeric Phosphorothioate Oligonuclootides

[2′-O-(2-methoxyethyl)]-[2′-deoxy]-[-2′-O-(methoxyethyl)] chimeric phosphorothioate oligonucleotides were prepared as per the procedure above for the 2′-O-methyl chimeric oligonucleotide, with the substitution of 2′-O-(methoxyethyl) amidites for the 2′-O-methyl amidites.

### [2′-O-(2-Methoxyethyl)Phosphodiester]-[2′-deoxy Phosphorothioatel-[2′-O-(2-Methoxyethyl) Phosphodiester] Chimeric Oligonucleotides

[2′-O-(2-methoxyethyl phosphodiester]-[2′-deoxy phosphorothioate]-[2′-O-(methoxyethyl) phosphodiester] chimeric oligonucleotides are prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065, herein incorporated by reference.

### Example 5

Design and Screening of Duplexed Antisense Compounds Targeting RANKL

In accordance with the present invention, a series of nucleic acid duplexes comprising the antisense compounds of the present invention and their complements can be designed to target RANKL. The nucleobase sequence of the antisense strand of the duplex comprises at least a portion of an oligonucleotide in Table 1. The ends of the strands can be modified by the addition of one or more natural or modified nucleobases to form an overhang. The sense strand of the dsRNA is then designed and synthesized as the complement of the antisense strand and can also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense compounds are evaluated for their ability to modulate RANKL expression.

When cells reached 80% confluency, they are treated with duplexed antisense compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense compound at a final concentration of 200 nM. After 5 hours of treatment, the medium is replaced with fresh medium. Cells are harvested 16 hours after treatment, at which time RNA is isolated and target reduction measured by RT-PCR.

### Example 6

Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the -16 amu product (±32±48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 7

Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 8

Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the compounds utilizing electrospray-mass spectroscopy. All assay test plates were diluted from the master plate using single and multi-channel robotic pipettors. Plates were judged to be acceptable if at least 85% of the compounds on the plate were at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells can be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

C2C12 Cells:

The mouse myoblast cell line was obtained from the American Type Culture Collection (ATCC) (Manassas, Va.). C2C12 cells were routinely cultured in DMEM, high glucose media (Invitrogen Corporation, Carlsbad, Calif.) supplemented with 10% fetal bovine serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 24-well plates (Falcon-353047 at a density of ˜10,000 cells/cm² for use in antisense oligonucleotide transfection.

UMR-106 Cells:

The rat osteosarcoma cell line was obtained from the American Type Culture Collection (ATCC) (Manassas, Va.). UMR-106 cells were routinely cultured in DMEM/F12 media (Invitrogen Corporation, Carlsbad, Calif.) supplemented with 10% fetal bovine serum (Invitrogen Corporation, Carlsbad, Calif.), 50ug/ml Gentamicin Sulfate Solution (Irvine Scientific, Santa Ana, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded onto 24-well plates (Falcon-353047) at a density of -5000 cells/cm² for use in antisense oligonucleotide transfection.

Primary Mouse Osteoblasts:

Primary mouse osteoblasts were prepared from calvaria of neonatal mice purchased from Charles River Laboratories. Primary mouse osteoblasts were routinely cultured in DMEM basal media (Invitrogen Corporation, Carlsbad, Calif.) supplemented with 10% fetal bovine serum (Invitrogen Corporation, Carlsbad, Calif.), 100 units per mL penicillin and 100 micrograms per mL streptomycin (Invitrogen Corporation, Carlsbad, Calif.). Cells were not passaged. Cells were seeded onto 6-well plates (Falcon-Primaria #353846) at a density of 50,000 cells/well for use in antisense oligonucleotide transfection.

Primary Mouse Bone Marrow-Derived Osteoclasts:

Primary mouse osteoclasts were prepared from the bone marrow of ˜4-month old, female BALB/C mice purchased from Charles River Laboratories. Primary mouse bone marrow suspensions were routinely cultured in alpha-MEM media (Invitrogen Corporation, Carlsbad, Calif.) supplemented with 10% Fetal Bovine Serum (Cat #SH30071.03) (Hyclone, Logan, Utah), 50 ug/ml Gentamicin Sulfate Solution (Irvine Scientific, Santa Ana, Calif.), 50ng/ml murine monocyte-colony stimulating factor (M-CSF) (R&D Systems, Minneapolis, Minn.) and 100 ng/ml soluble human receptor activator of NF-kB ligand (shRANKL) (Peprotech, Rocky Hill, N.J.). Cells were seeded onto 24-well plates (Falcon-353047) at a density of ˜75,000 cells/cm² for use in antisense oligonucleotide transfection.

Treatment with Antisense Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO: 1) which is targeted to human H-ras, or ISIS 18078, (GTGCGCGCGAGCCCGAAATC, SEQ ID NO: 2) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO: 3, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 10

Analysis of Oligonucleotid Inhibition of RANKL Expression

Antisense modulation of RANKL expression can be assayed in a variety of ways known in the art. For example, RANKL mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. The preferred method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of RANKL can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to RANKL can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 11

Design of Phenotypic Assays and in vivo Studies for the use of RANKL Inhibitors

Phenotypic Assays

Once RANKL inhibitors have been identified by the methods disclosed herein, the compounds are further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of RANKL in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with RANKL inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the gennotype of the cell (measurement of the expression of one or more of the genes of the cell) after treatment is also used as an indicator of the efficacy or potency of the RANKL inhibitors. Hallmark genes, or those genes suspected to be associated with a specific disease state, condition, or phenotype, are measured in both treated and untreated cells.

In vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study. To account for the psychological effects of receiving treatments, volunteers are randomly given placebo or RANKL inhibitor. Furthermore, to prevent the doctors from being biased in treatments, they are not informed as to whether the medication they are administering is a RANKL inhibitor or a placebo. Using this randomization approach, each volunteer has the same chance of being given either the new treatment or the placebo.

Volunteers receive either the RANKL inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding RANKL or RANKL protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and RANKL inhibitor treatment. In general, the volunteers treated with placebo have little or no response to treatment, whereas the volunteers treated with the RANKL inhibitor show positive trends in their disease state or condition index at the conclusion of the study.

### Example 12

RNA Isolation

Poly(A)+mRNA Isolation

Poly(A)+mRNA was isolated according to Miura et al., (Clin. Chem., 1996, 42, 1758-1764). Other methods for poly(A)+mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates can be treated similarly, using appropriate volumes of all solutions.

Total RNA Isolation

Total RNA was isolated using an RNEASY ₉₆Tm kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY ₉₆™ plate and the vacuum was applied for 2 minutes. 1 mL of Buffer RPE was then added to each well of the RNEASY ₉₆™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps can be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-time Quantitative PCR Analysis of RANKL mRNA Levels

Quantitation of RANKL mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of DATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485nm and emission at 530 nm.

Probes and primers to human RANKL were designed to hybridize to a human RANKL sequence, using published sequence information (GenBank accession number AF053712.1, incorporated herein as SEQ ID NO:4). For human RANKL the PCR primers were:
forward primer: CCTAGCTACAGAGTATCTTCAACTAATGGT (SEQ ID NO: 5)
reverse primer: TGGTGCTTCCTCCTTTCATCA (SEQ ID NO: 6) and the
PCR probe was: FAM-CGTCACTAAAACCAGCATCAAAATCCCAAGT-TAMRA (SEQ ID NO: 7) where FAM is the fluorescent dye and TAMRA is the quencher dye. For human GAPDH the PCR primers were:
forward primer: GAAGGTGAAGGTCGGAGTC(SEQ ID NO:8)
reverse primer: GAAGATGGTGATGGGATTTC (SEQ ID NO:9) and the
PCR probe was: 5′ JOE-CAAGCTTCCCGTTCTCAGCC-TAMRA 3′ (SEQ ID NO: 10) where JOE is the fluorescent reporter dye and TAMRA is the quencher dye.

Probes and primers to mouse RANKL were designed to hybridize to a mouse RANKL sequence, using published sequence information (GenBank accession number AF013170.1, incorporated herein as SEQ ID NO:ll). For mouse RANKL the PCR primers were:
forward primer: TGCAGCATCGCTCTGTTCC (SEQ ID NO:12)
reverse primer: AAGCAGTGAGTGCTGTCTTCTGA (SEQ ID NO: 13) and
the PCR probe was: FAM-TTTCGAGCGCAGATGGATCCTAACAGAA-TAMRA (SEQ ID NO: 14) where FAM is the fluorescent reporter dye and TAMRA is the quencher dye. For mouse GAPDH the PCR primers were:
forward primer: GGCAAATTCAACGGCACAGT(SEQ ID NO:15)
reverse primer: GGGTCTCGCTCCTGGAAGAT(SEQ ID NO:16) and the
PCR probe was: 5′ JOE-AAGGCCGAGAATGGGAAGCTTGTCATC-TAMRA 3′ (SEQ ID NO: 17) where JOE is the fluorescent reporter dye and TAMRA is the quencher dye.

Probes and primers to rat RANKL were designed to hybridize to a rat RANKL sequence, using published sequence information (GenBank accession number NM_057149.1, incorporated herein as SEQ ID NO:18). For rat RANKL the PCR primers were:
forward primer: TTTATTCCATAAACGTTGGAGGATT (SEQ ID NO:19)
reverse primer: TTGGACACCTGGACGCTAATT (SEQ ID NO: 20) and the
PCR probe was: FAM-TTCAAGCTCCGGGCTGGTGAGG-TAMRA (SEQ ID NO: 21) where FAM is the fluorescent reporter dye and TAMRA is the quencher dye. For rat GAPDH the PCR primers were:
forward primer: TGTTCTAGAGACAGCCGCATCTT(SEQ ID NO:22)
reverse primer: CACCGACCTTCACCATCTTGT(SEQ ID NO:23) and the
PCR probe was: 5′ JOE-TTGTGCAGTGCCAGCCTCGTCTCA-TAMRA 3′ (SEQ ID NO: 24) where JOE is the fluorescent reporter dye and TAMRA is the quencher dye.

### Example 14

Northern Blot Analysis of RANKL mRNA Levels

Eighteen hours after antisense treatment, cell monolayers were washed twice with cold PBS and lysed in 1 mL RNAZOLT (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human RANKL, a human RANKL specific probe was prepared by PCR using the forward primer CCTAGCTACAGAGTATCTTCAACTAATGGT (SEQ ID NO: 5) and the reverse primer TGGTGCTTCCTCCTTTCATCA (SEQ ID NO: 6). To normalize for variations in loading and transfer efficiency membranes were stripped and probed for human glyceraldehyde-3-phosphate dehydrogenase (GAPDH) RNA (Clontech, Palo Alto, Calif.).

To detect mouse RANKL, a mouse RANKL specific probe was prepared by PCR using the forward primer TGCAGCATCGCTCTGTTCC (SEQ ID NO: 12) and the reverse primer AAGCAGTGAGTGCTGTCTTCTGA (SEQ ID NO: 13). To normalize for variations in loading and transfer efficiency membranes were stripped and probed for mouse glyceraldehyde-3-phosphate dehydrogenase (GAPDH) RNA (Clontech, Palo Alto, Calif.).

To detect rat RANKL, a rat RANKL specific probe was prepared by PCR using the forward primer TTTATTCCATAAACGTTGGAGGATT (SEQ ID NO: 12) and the reverse primer TTGGACACCTGGACGCTAATT (SEQ ID NO: 13). To normalize for variations in loading and transfer efficiency membranes were stripped and probed for rat glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 15

Antisonse Inhibition of Human RANeL Expression by Chimeric Phosphorothioate Oligonuclootides having 2′-MOE Wings and a Deoxy Gap

In accordance with the present invention, a series of antisense compounds was designed to target different regions of the human RANKL RNA, using published sequences (GenBank accession number AF053712.1, incorporated herein as SEQ ID NO: 4). The compounds are shown in Table 1. “Target site” indicates the first (5′-most) nucleotide number on the particular target sequence to which the compound binds. All compounds -in Table 1 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds can be analyzed for their effect on human RANKL mRNA levels by quantitative real-time PCR as described in other examples herein.

TABLE 1

Inhibition of human RANKL mRNA levels by chimeric
phosphorothioate oligonucleotides having 2′-MOE
wings and a
deoxy gap

| | | |
|---|---|---|
| TARGET | | |
| SEQ ID | TARGET | SEQ ID |

| | | | | | |
|---|---|---|---|---|---|
| ISIS # | REGION | NO | SITE | SEQUENCE | NO |

| | | | | | |
|---|---|---|---|---|---|
| 109549 | 5′UTR | 4 | 1 | ccgagctcggtaccaagctt | 25 |
| 109550 | 5′UTR | 4 | 26 | cggacgcgtgggtcgagtag | 26 |
| 109551 | 5′UTR | 4 | 103 | cggccaactccggaggctgc | 27 |
| 109552 | 5′UTR | 4 | 129 | ctcccgctccctccccttct | 28 |
| 109553 | 5′UTR | 4 | 154 | ctctcgcttcggagctctcc | 29 |
| 109554 | Start Codon | 4 | 166 | tggcgctcggccctctcgct | 30 |
| 109555 | Start Codon | 4 | 175 | cgcggcgcatggcgctcggc | 31 |
| 109556 | Start Codon | 4 | 184 | ctctgctggcgcggcgcatg | 32 |
| 109557 | Coding | 4 | 209 | gagccacgcaggtacttggt | 33 |
| 109558 | Coding | 4 | 285 | ctggtgcggcgcaggcggcg | 34 |
| 109559 | Coding | 4 | 332 | cccagccccaggagggccac | 35 |
| 109560 | Coding | 4 | 356 | acgctgcagacaacctggcc | 36 |
| 109561 | Coding | 4 | 381 | cgctctgaaatagaagaaca | 37 |
| 109562 | Coding | 4 | 407 | tctgatattctattaggatc | 38 |
| 109563 | Coding | 4 | 436 | aaattctataaatgcagtga | 39 |
| 109564 | Coding | 4 | 450 | ttcatggagtctcaaaattc | 40 |
| 109565 | Coding | 4 | 463 | gaaaatctgcattttcatgg | 41 |
| 109566 | Coding | 4 | 476 | agagttgtgtcttgaaaatc | 42 |
| 109567 | Coding | 4 | 487 | cttgactctccagagttgtg | 43 |
| 109568 | Coding | 4 | 509 | gaatcaggtattaattttgt | 44 |
| 109569 | Coding | 4 | 519 | tctcctacatgaatcaggta | 45 |
| 109570 | Coding | 4 | 532 | aggcctgtttaattctccta | 46 |
| 109571 | Coding | 4 | 555 | ttccttttgcacagctcctt | 47 |
| 109572 | Coding | 4 | 575 | gatccaacgatatgttgtaa | 48 |
| 109573 | Coding | 4 | 598 | ctttctctgctctgatgtgc | 49 |
| 109574 | Coding | 4 | 621 | taaccatgagccatccacca | 50 |
| 109575 | Coding | 4 | 643 | gcttgctcctcttggccaga | 51 |
| 109576 | Coding | 4 | 666 | atgagcaaaaggctgagctt | 52 |
| 109577 | Coding | 4 | 690 | gatgtcggtggcattaatag | 53 |
| 109578 | Coding | 4 | 714 | actcactttatgggaaccag | 54 |
| 109579 | Coding | 4 | 737 | cgatcatggtaccaagagga | 55 |
| 109580 | Coding | 4 | 760 | tgttggagatcttggcccaa | 56 |
| 109581 | Coding | 4 | 782 | agttttccattgctaaaagt | 57 |
| 109582 | Coding | 4 | 791 | ttaactattagttttccatt | 58 |
| 109583 | Coding | 4 | 805 | aaaagccatcctgattaact | 59 |
| 109584 | Coding | 4 | 827 | caaatgttggcatacaggta | 60 |
| 109585 | Coding | 4 | 849 | tgaagtttcatgatgtcgaa | 61 |
| 109586 | Coding | 4 | 871 | gatactctgtagctaggtct | 62 |
| 109587 | Coding | 4 | 894 | agtgacgtacaccattagtt | 63 |
| 109588 | Coding | 4 | 919 | aacttgggattttgatgctg | 64 |
| 109589 | Coding | 4 | 941 | cctcctttcatcagggtatg | 65 |
| 109590 | Coding | 4 | 965 | ttccctgaccaatacttggt | 66 |
| 109591 | Coding | 4 | 1004 | aatccaccaacgtttatgga | 67 |
| 109592 | Coding | 4 | 1044 | gacctcgatgctgatttcct | 68 |
| 109593 | Coding | 4 | 1083 | tgttgcatcctgatccggat | 69 |
| 109594 | Coding | 4 | 1107 | tcgaactttaaaagccccaa | 70 |
| 109595 | 3′UTR | 4 | 1155 | catccaggaaatacataaca | 71 |
| 109596 | 3′UTR | 4 | 1195 | tatacatctttcttggcttg | 72 |
| 109597 | 3′UTR | 4 | 1222 | atgcctcttagtagtctcac | 73 |
| 109598 | 3′UTR | 4 | 1263 | aggtcaagagcatggatact | 74 |
| 109599 | 3′UTR | 4 | 1287 | ctgtaaatacgcgtgttctc | 75 |
| 109600 | 3′UTR | 4 | 1310 | atgagtctaacatctcccac | 76 |
| 109601 | 3′UTR | 4 | 1349 | aattcattacaaaatttaaa | 77 |
| 109602 | 3′UTR | 4 | 1370 | ccaatctggtttaattctag | 78 |
| 109603 | 3′UTR | 4 | 1392 | ataaggtcaacccgtaattg | 79 |
| 109604 | 3′UTR | 4 | 1414 | catagcccacatgcagtttc | 80 |
| 109605 | 3′UTR | 4 | 1437 | catgaccagggaccaacccc | 81 |
| 109606 | 3′UTR | 4 | 1475 | ctagatgacaccctctccac | 82 |
| 109607 | 3′UTR | 4 | 1497 | ttcagatgatccttcaattg | 83 |
| 109608 | 3′UTR | 4 | 1519 | acaattcaaaagaatttgcc | 84 |
| 109609 | 3′UTR | 4 | 1539 | gcaggttccagcatgatgta | 85 |
| 109610 | 3′UTR | 4 | 1604 | atataactttagatattata | 86 |
| 109611 | 3′UTR | 4 | 1630 | tttgcaaagaaaacattaca | 87 |
| 109612 | 3′UTR | 4 | 1653 | agcacaaatataatttacaa | 88 |
| 109613 | 3′UTR | 4 | 1678 | ttaaatattttgaatcaaat | 89 |
| 109614 | 3′UTR | 4 | 1716 | acatttaaaacattaaatat | 90 |
| 109615 | 3′UTR | 4 | 1738 | tgcaccagttaaatatgtct | 91 |
| 109616 | 3′UTR | 4 | 1761 | ttttccccagggaatttaca | 92 |
| 109617 | 3′UTR | 4 | 1803 | gatattaggaaacaacattt | 93 |
| 109618 | 3′UTR | 4 | 1822 | gaagaaatatactgcatttg | 94 |
| 109619 | 3′UTR | 4 | 1828 | aagaacgaagaaatatactg | 95 |
| 109620 | 3′UTR | 4 | 1899 | tattattcaaggcatccatt | 96 |
| 109621 | 3′UTR | 4 | 1920 | cctggtggccaacatcctgc | 97 |
| 109622 | 3′UTR | 4 | 1942 | tagtttctaaatttgaaagg | 98 |
| 109623 | 3′UTR | 4 | 1966 | caatgtcagctttctaaagt | 99 |
| 109624 | 3′UTR | 4 | 2004 | ttgacagatttcagtggccc | 100 |
| 109625 | 3′UTR | 4 | 2027 | gttcaacaattatataacta | 101 |
| 109626 | 3′UTR | 4 | 2094 | actaataacttttctatttt | 102 |
| 109627 | 3′UTR | 4 | 2136 | aacatttactaaattaaaat | 103 |
| 109628 | 3′UTR | 4 | 2175 | aacattcaaaggcaatgttt | 104 |
| 181859 | 5′UTR | 4 | 3 | atccgagctcggtaccaagc | 105 |
| 181862 | 5′UTR | 4 | 27 | gcggacgcgtgggtcgagta | 106 |
| 181864 | 5′UTR | 4 | 52 | agcccggctttggctcctgg | 107 |
| 181866 | 5′UTR | 4 | 94 | ccggaggctgcggcggagcc | 108 |
| 181868 | 5′UTR | 4 | 102 | ggccaactccggaggctgcg | 109 |
| 181870 | 5′UTR | 4 | 150 | cgcttcggagctctcctccc | 110 |
| 181872 | Start Codon | 4 | 167 | atggcgctcggccctctcgc | 111 |
| 181874 | Start Codon | 4 | 169 | gcatggcgctcggccctctc | 112 |
| 181876 | Start Codon | 4 | 173 | cggcgcatggcgctcggccc | 113 |
| 181878 | Start Codon | 4 | 185 | tctctgctggcgcggcgcat | 114 |
| 181881 | Coding | 4 | 219 | catctcctccgagccacgca | 115 |
| 181883 | Coding | 4 | 288 | gggctggtgcggcgcaggcg | 116 |
| 181885 | Coding | 4 | 312 | gaacatggagcgggaggcgg | 117 |
| 181887 | Coding | 4 | 341 | tggcccagccccagccccag | 118 |
| 181889 | Coding | 4 | 343 | cctggcccagccccagcccc | 119 |
| 181891 | Coding | 4 | 411 | atcttctgatattctattag | 120 |
| 181893 | Coding | 4 | 427 | aaatgcagtgagtgccatct | 121 |
| 181895 | Coding | 4 | 547 | gcacagctccttgaaaggcc | 122 |
| 181897 | Coding | 4 | 615 | tgagccatccaccatcgctt | 123 |
| 181899 | Coding | 4 | 617 | catgagccatccaccatcgc | 124 |
| 181901 | Coding | 4 | 700 | aaccagatgggatgtcggtg | 125 |
| 181903 | Coding | 4 | 748 | tggcccaaccccgatcatgg | 126 |
| 181905 | Coding | 4 | 834 | tcgaaagcaaatgttggcat | 127 |
| 181907 | Coding | 4 | 956 | caatacttggtgcttcctcc | 128 |
| 181909 | Coding | 4 | 1051 | ggttggagacctcgatgctg | 129 |
| 181911 | Coding | 4 | 1080 | tgcatcctgatccggatcca | 130 |
| 181913 | Coding | 4 | 1116 | atctatatctcgaactttaa | 131 |
| 181915 | Stop Codon | 4 | 1119 | tcaatctatatctcgaactt | 132 |
| 181917 | 3′UTR | 4 | 1160 | ccaaacatccaggaaataca | 133 |
| 181919 | 3′UTR | 4 | 1189 | tctttcttggcttgttttaa | 134 |
| 181921 | 3′UTR | 4 | 1245 | ctgagtcgtgtaccgttggg | 135 |
| 181923 | 3′UTR | 4 | 1295 | cccactggctgtaaatacgc | 136 |
| 181925 | 3′UTR | 4 | 1382 | cccgtaattgctccaatctg | 137 |
| 181927 | 3′UTR | 4 | 1424 | caacccctcccatagcccac | 138 |
| 181930 | 3′UTR | 4 | 1481 | attgcgctagatgacaccct | 139 |
| 181932 | 3′UTR | 4 | 1487 | ccttcaattgcgctagatga | 140 |
| 181934 | 3′UTR | 4 | 1513 | caaaagaatttgccccttca | 141 |
| 181936 | 3′UTR | 4 | 1610 | tctgaaatataactttagat | 142 |
| 181938 | 3′UTR | 4 | 1617 | cattacatctgaaatataac | 143 |
| 181940 | 3′UTR | 4 | 1670 | tttgaatcaaatactatagc | 144 |
| 181942 | 3′UTR | 4 | 1721 | tctgtacatttaaaacatta | 145 |
| 181944 | 3′UTR | 4 | 1744 | acaaagtgcaccagttaaat | 146 |
| 181946 | 3′UTR | 4 | 1758 | tccccagggaatttacaaag | 147 |
| 181948 | 3′UTR | 4 | 1770 | agctgcaagttttccccagg | 148 |
| 181950 | 3′UTR | 4 | 1867 | cacaggcttgacaagtctga | 149 |
| 181952 | 3′UTR | 4 | 1921 | acctggtggccaacatcctg | 150 |
| 181954 | 3′UTR | 4 | 1937 | tctaaatttgaaaggcacct | 151 |
| 181956 | 3′UTR | 4 | 1992 | agtggcccattatgtatcct | 152 |
| 181958 | 3′UTR | 4 | 2048 | ggcacttgtggaaaaacacc | 153 |
| 181960 | 3′UTR | 4 | 2109 | ttttgctgataaaccactaa | 154 |

### Example 16

Antisense Inhibition of Mouse RANKL Expression by Chimeric Phosphorothioate Oligonucleotides having 2′-MOE Wings and a Deoxy Gap.

In accordance with the present invention, a second series of antisense compounds was designed to target different regions of the mouse RANKL RNA, using published sequences (GenBank accession number AF013170.1, incorporated herein as SEQ ID NO: 11). The compounds are shown in Table 2. “Target site” indicates the first (5′-most) nucleotide number on the particular target nucleic acid to which the compound binds. All compounds in Table 2 are chimeric oligonucleotides (“gapmers”) 20 nucleotides in length, composed of a central “gap” region consisting of ten 2′-otides, deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines. The compounds were analyzed for their effect on mouse RANKL mRNA levels by quantitative real-time PCR as described in other examples herein. Data are averages from three experiments in which C2C12 cells were treated with the antisense oligonucleotides of the present invention. If present “N.D.” indicates “no data”.

TABLE 2

Inhibition of mouse RANKL mRNA levels by chimeric
phosphorothioate oligonucleotides having 2′-MOE wings and a
deoxy gap

| | | | |
|---|---|---|---|
| TARGET | | | |
| SEQ ID | TARGET | % | SEQ ID |

| | | | | | | |
|---|---|---|---|---|---|---|
| ISIS # | REGION | NO | SITE | SEQUENCE | INHIB | NO |

| | | | | | | |
|---|---|---|---|---|---|---|
| 180788 | 5′UTR | 11 | 20 | gttcacaaaggtcctggcag | 62 | 155 |
| 180789 | 5′UTR | 11 | 51 | gcagactccgccacggcccc | 34 | 156 |
| 180790 | 5′UTR | 11 | 52 | agcagactccgccacggccc | 59 | 157 |
| 180791 | 5′UTR | 11 | 58 | ccgccgagcagactccgcca | 44 | 158 |
| 180792 | 5′UTR | 11 | 95 | tgctccgcgatcgttctctc | 77 | 159 |
| 180793 | 5′UTR | 11 | 99 | gccctgctccgcgatcgttc | 74 | 160 |
| 180794 | Start Codon | 11 | 131 | ccggcgcatggcgcggcgcc | 0 | 161 |
| 180795 | Start Codon | 11 | 137 | gctggcccggcgcatggcgc | 62 | 162 |
| 180796 | Start Codon | 11 | 140 | tcggctggcccggcgcatgg | 67 | 163 |
| 180797 | Start Codon | 11 | 142 | tctcggctggcccggcgcat | 60 | 164 |
| 180798 | Coding | 11 | 205 | ccttcgtgtgggacgccggg | 34 | 165 |
| 180799 | Coding | 11 | 262 | cgggaggeggcgggtggcgg | 52 | 166 |
| 180800 | Coding | 11 | 270 | acatggagcgggaggcggcg | 54 | 167 |
| 180801 | Coding | 11 | 271 | aacatggagcgggaggcggc | 40 | 168 |
| 180802 | Coding | 11 | 340 | gctcgaaagtacaggaacag | 79 | 169 |
| 180803 | Coding | 11 | 448 | tcttcactctccagagtcga | 55 | 170 |
| 180804 | Coding | 11 | 512 | cagttccttctgcacggccc | 44 | 171 |
| 180805 | Coding | 11 | 598 | ggcttgcctcgctgggccac | 62 | 172 |
| 180807 | Coding | 11 | 783 | agcaaatgttggcgtacagg | 74 | 173 |
| 180808 | Coding | 11 | 832 | agctgaagatagtctgtagg | 74 | 174 |
| 180809 | Coding | 11 | 867 | ggattttgatgctggtttta | 53 | 175 |
| 180810 | Coding | 11 | 925 | tcagaattgcccgaccagtt | 83 | 176 |
| 180811 | Coding | 11 | 991 | atgctaatttcttcaccagc | 67 | 177 |
| 180812 | Coding | 11 | 1024 | tccggatccagcagggaagg | 47 | 178 |
| 180813 | Stop Codon | 11 | 1092 | atgttccacgaaatgagtct | 48 | 179 |
| 180814 | 3′UTR | 11 | 1159 | gtcttacacatgtatagaca | 66 | 180 |
| 180815 | 3′UTR | 11 | 1188 | tcatacaccgtgggccatgt | 30 | 181 |
| 180816 | 3′UTR | 11 | 1278 | accgttgtgtaatcaccatg | 35 | 182 |
| 180817 | 3′UTR | 11 | 1337 | gcatcggaatacctctccca | 51 | 183 |
| 180818 | 3′UTR | 11 | 1411 | tcagtggcacatgtccaggg | 59 | 184 |
| 180819 | 3′UTR | 11 | 1507 | tgcaggtcccagcgcaatgt | 66 | 185 |
| 180820 | 3′UTR | 11 | 1513 | cttatttgcaggtcccagcg | 44 | 186 |
| 180821 | 3′UTR | 11 | 1586 | cattacacctgaaatataac | 15 | 187 |
| 180822 | 3′UTR | 11 | 1635 | aatcaaatactatagcacaa | 24 | 188 |
| 180823 | 3′UTR | 11 | 1646 | taaatattttgaatcaaata | 9 | 189 |
| 180824 | 3′UTR | 11 | 1666 | tgtcaacagtgagacatttt | 29 | 190 |
| 180825 | 3′UTR | 11 | 1687 | gtacatttaaaacattaaat | 25 | 191 |
| 180826 | 3′UTR | 11 | 1714 | tacaaagtgcaccagttaaa | 33 | 192 |
| 180827 | 3′UTR | 11 | 1738 | agctacgagtaccttcaggg | 54 | 193 |
| 180828 | 3′UTR | 11 | 1843 | tttattttgcttgcatagtt | 44 | 194 |
| 180829 | 3′UTR | 11 | 1903 | aaggcacctggtgaccaaca | 23 | 195 |
| 180830 | 3′UTR | 11 | 1906 | tgaaaggcacctggtgacca | 5 | 196 |
| 180831 | 3′UTR | 11 | 1980 | cctgacagatttcagtagcc | 54 | 197 |
| 180832 | 3′UTR | 11 | 2057 | ggaaaaaaaagaaaccaaaa | 0 | 198 |
| 180833 | 3′UTR | 11 | 2156 | cagagacaatgcttttattg | 22 | 199 |

As shown in Table 2, SEQ ID NOs 155, 157, 158, 159, 160, 162, 163, 164, 166, 167, 168, 169, 170, 171, 172, 173, 174, 175, 176, 177, 178, 179, 180, 183, 184, 185, 186, 193, 194, and 197 demonstrated at least 40% inhibition of mouse RANKL expression in this experiment and are therefore preferred. More preferred are SEQ ID Nos 180 and 185. The target regions to which these preferred sequences are complementary are herein referred to as “preferred target segments” and are therefore preferred for targeting by compounds of the present invention. These preferred target segments are shown in Table 4. These sequences are shown to contain thymine (T) but one of skill in the art will appreciate that thymine (T) is generally replaced by uracil (U) in RNA sequences. The sequences represent the reverse complement of the preferred antisense compounds shown in Table 2. “Target site” indicates the first (5′-most) nucleotide number on the particular target nucleic acid to which the oligonucleotide binds. Also shown in Table 4 is the species in which each of the preferred target segments was found.

### Example 17

Antisense Inhibition of rat RANKL Expression by Chimeric Phosphorothioate Oligonucleotides having 21-MOE Wings and a Deoxy Gap.

In accordance with the present invention, a second series of antisense compounds was designed to target different regions of the rat RANKL RNA, using published sequences (GenBank accession number NM_{—}057149.1, incorporated herein as SEQ ID NO: 18, and GenBank accession number AF425669.1, incorporated herein as SEQ ID NO: 200). The compounds are shown in Table 3. “Target site” indicates the first (5′-most) nucleotide number on the particular target nucleic acid to which the compound binds. All compounds in Table 3 are chimeric oligonucleotides (“gapmers”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P=S) throughout the oligonucleotide. All cytidine residues are 5-methylcytidines. The compounds were analyzed for their effect on rat RANKL mRNA levels by quantitative real-time PCR as described in other examples herein. Data are averages from three experiments in which UMR-106 cells were treated with the antisense oligonucleotides of the present invention. The positive control for each datapoint is identified in the table by sequence ID number. If present, “N.D.” indicates “no data”.

TABLE 3

Inhibition of rat RANKL mRNA levels by chimeric
phosphorothioate oligonucleotides having 2′-MOE wings and a
deoxy gap

| | | | | |
|---|---|---|---|---|
| TARGET | | | | CONTROL |
| SEQ ID | TARGET | % | SEQ ID | SEQ ID |

| | | | | | | | |
|---|---|---|---|---|---|---|---|
| ISIS # | REGION | NO | SITE | SEQUENCE | INHIB | NO | NO |

| | | | | | | | |
|---|---|---|---|---|---|---|---|
| 180802 | Coding | 18 | 199 | gctcgaaagtacaggaacag | 40 | 169 | 1 |
| 180806 | Coding | 18 | 503 | ggcagcattgatggtgaggt | 9 | 201 | 1 |
| 180807 | Coding | 18 | 648 | agcaaatgttggcgtacagg | 48 | 173 | 1 |
| 180809 | Coding | 18 | 732 | ggattttgatgctggtttta | 57 | 175 | 1 |
| 286586 | Coding | 18 | 15 | ggtacttgccgtagtctcgg | 63 | 202 | 1 |
| 286587 | Coding | 18 | 20 | gcgcaggtacttgccgtagt | 50 | 203 | 1 |
| 286588 | Coding | 18 | 178 | gcgatgctgcagaccacctg | 63 | 204 | 1 |
| 286589 | Coding | 18 | 183 | acagagcgatgctgcagacc | 49 | 205 | 1 |
| 286590 | Coding | 18 | 188 | caggaacagagcgatgctgc | 47 | 206 | 1 |
| 286591 | Coding | 18 | 193 | aagtacaggaacagagcgat | 17 | 207 | 1 |
| 286592 | Coding | 18 | 198 | ctcgaaagtacaggaacaga | 16 | 208 | 1 |
| 286593 | Coding | 18 | 203 | ctgcgctcgaaagtacagga | 49 | 209 | 1 |
| 286594 | Coding | 18 | 208 | tccatctgcgctcgaaagta | 35 | 210 | 1 |
| 286595 | Coding | 18 | 213 | taggatccatctgcgctcga | 46 | 211 | 1 |
| 286596 | Coding | 18 | 218 | tctgttaggatccatctgcg | 58 | 212 | 1 |
| 286597 | Coding | 18 | 223 | gatattctgttaggatccat | 57 | 213 | 1 |
| 286598 | Coding | 18 | 228 | cttctgatattctgttagga | 6 | 214 | 1 |
| 286599 | Coding | 18 | 238 | cgcgtgctgtcttctgatat | 46 | 215 | 1 |
| 286600 | Coding | 18 | 252 | ttctgtagaagcagcgcgtg | 13 | 216 | 1 |
| 286601 | Coding | 18 | 268 | tcacggagtctcagaattct | 63 | 217 | 1 |
| 286602 | Coding | 18 | 279 | aacctgtattttcacggagt | 48 | 218 | 1 |
| 286603 | Coding | 18 | 293 | agtcgagtcctgcaaacctg | 59 | 219 | 1 |
| 286604 | Coding | 18 | 298 | tccagagtcgagtcctgcaa | 68 | 220 | 1 |
| 286605 | Coding | 18 | 369 | tttgcacggccccttgaaag | 53 | 221 | 1 |
| 286606 | Coding | 18 | 379 | tgtaattccctttgcacggc | 37 | 222 | 1 |
| 286607 | Coding | 18 | 397 | tgtggccccacaatgtgttg | 48 | 223 | 1 |
| 286608 | Coding | 18 | 480 | caaacggctgagcctcaggc | 25 | 224 | 1 |
| 286609 | Coding | 18 | 502 | gcagcattgatggtgaggtg | 17 | 225 | 1 |
| 286610 | Coding | 18 | 529 | actttatgggaacccgatgg | 34 | 226 | 1 |
| 286611 | Coding | 18 | 542 | agaggacagactgactttat | 47 | 227 | 1 |
| 286612 | Coding | 18 | 550 | tggtaccaagaggacagact | 5 | 228 | 1 |
| 286613 | Coding | 18 | 555 | gatcatggtaccaagaggac | 39 | 229 | 1 |
| 286614 | Coding | 18 | 571 | atcttggcccagcctcgatc | 41 | 230 | 1 |
| 286615 | Coding | 18 | 576 | tagagatcttggcccagcct | 45 | 231 | 1 |
| 286616 | Coding | 18 | 581 | catgttagagatcttggccc | 31 | 232 | 1 |
| 286617 | Coding | 18 | 601 | agttttccgttgcttaacgt | 52 | 233 | 1 |
| 286618 | Coding | 18 | 607 | acccttagttttccgttgct | 57 | 234 | 1 |
| 286619 | Coding | 18 | 612 | ggttaacccttagttttccg | 61 | 235 | 1 |
| 286620 | Coding | 18 | 617 | atcttggttaacccttagtt | 48 | 236 | 1 |
| 286621 | Coding | 18 | 622 | aagccatcttggttaaccct | 33 | 237 | 1 |
| 286622 | Coding | 18 | 627 | aatagaagccatcttggtta | 12 | 238 | 1 |
| 286623 | Coding | 18 | 628 | taatagaagccatcttggtt | 16 | 239 | 1 |
| 286624 | Coding | 18 | 632 | caggtaatagaagccatctt | 22 | 240 | 1 |
| 286625 | Coding | 18 | 637 | gcgtacaggtaatagaagcc | 50 | 241 | 1 |
| 286626 | Coding | 18 | 642 | tgttggcgtacaggtaatag | 41 | 242 | 1 |
| 286627 | Coding | 18 | 647 | gcaaatgttggcgtacaggt | 53 | 243 | 1 |
| 286628 | Coding | 18 | 691 | agatagtccgcaggtacgct | 24 | 244 | 1 |
| 286629 | Coding | 18 | 703 | accatcagctgaagatagtc | 63 | 245 | 1 |
| 286630 | Coding | 18 | 717 | ttttaacgacatataccatc | 44 | 246 | 1 |
| 286631 | Coding | 18 | 724 | atgctggttttaacgacata | 35 | 247 | 1 |
| 286632 | Coding | 18 | 736 | cttgggattttgatgctggt | 51 | 248 | 1 |
| 286633 | Coding | 18 | 776 | ccagttcttagtgctccccc | 4 | 249 | 1 |
| 286634 | Coding | 18 | 793 | aattcagaattccctgacca | 21 | 250 | 1 |
| 286635 | Coding | 18 | 802 | taaaagtggaattcagaatt | 11 | 251 | 1 |
| 286636 | Coding | 18 | 807 | tggaataaaagtggaattca | 21 | 252 | 1 |
| 286637 | Coding | 18 | 899 | cgcatcttgatccggatcca | 27 | 253 | 1 |
| 286638 | Coding | 18 | 906 | agtacgtcgcatcttgatcc | 26 | 254 | 1 |
| 286639 | Coding | 18 | 911 | cccaaagtacgtcgcatctt | 48 | 255 | 1 |
| 286640 | Coding | 18 | 916 | aaagccccaaagtacgtcgc | 56 | 256 | 1 |
| 286641 | Coding | 18 | 921 | ctttgaaagccccaaagtac | 49 | 257 | 1 |
| 286642 | Stop Codon | 18 | 938 | tcagtctatgtcttgaactt | 41 | 258 | 1 |
| 286643 | 3′UTR | 200 | 178 | gcatatccatgctaaggctc | 69 | 259 | 1 |
| 286644 | 3′UTR | 200 | 231 | gtcttacacatgtatctaca | 57 | 260 | 1 |
| 286645 | 3′UTR | 200 | 238 | cttagtagtcttacacatgt | 51 | 261 | 1 |
| 286646 | 3′UTR | 200 | 262 | ttttgtacaacgtgggccac | 53 | 262 | 1 |
| 286647 | 3′UTR | 200 | 293 | gacctgtacagggtcgagag | 53 | 263 | 1 |
| 286648 | 3′UTR | 200 | 346 | ccattgtgtgatcaccatga | 72 | 264 | 1 |
| 286649 | 3′UTR | 200 | 463 | tagacccagagactatgtat | 25 | 265 | 1 |
| 286650 | 3′UTR | 200 | 482 | gtggcacaggcccaggagtt | 53 | 266 | 1 |
| 286651 | 3′UTR | 200 | 491 | aggttctcagtggcacaggc | 80 | 267 | 1 |
| 286652 | 3′UTR | 200 | 523 | ctctgcaatgtaacggtacc | 53 | 268 | 1 |
| 286653 | 3′UTR | 200 | 535 | aaaccatcatttctctgcaa | 58 | 269 | 1 |
| 286654 | 3′UTR | 200 | 588 | acttatttgcaggttccagc | 55 | 270 | 1 |
| 286655 | 3′UTR | 200 | 666 | gaaaaccttacacctgaaat | 0 | 271 | 1 |
| 286656 | 3′UTR | 200 | 675 | ttttgcacagaaaaccttac | 12 | 272 | 1 |
| 286657 | 3′UTR | 200 | 778 | ccagttaaatacatcttgac | 10 | 273 | 1 |

As shown in Table 3, SEQ ID NOs 169, 173, 175, 202, 203, 204, 205, 206, 209, 211, 212, 213, 215, 217, 218, 219, 220, 221, 223, 227, 230, 231, 233, 234, 235, 236, 241, 242, 243, 245, 246, 248, 255, 256, 257, 258, 259, 260, 261, 262, 263, 264, 266, 267, 268, 269, and 270 demonstrated at least 40% inhibition of rat RANKL expression in this experiment and are therefore preferred. More preferred are SEQ ID Nos 259, 264, and 267. The target regions to which these preferred sequences are complementary are herein referred to as “preferred target segments” and are therefore preferred for targeting by compounds of the present invention. These preferred target segments are shown in Table 4. These sequences are shown to contain thymine (T) but one of skill in the art will appreciate that thymine (T) is generally replaced by uracil (U) in RNA sequences. The sequences represent the reverse complement of the preferred antisense compounds shown in Tables 2 and 3. “Target site” indicates the first (5′-most) nucleotide number on the particular target nucleic acid to which the oligonucleotide binds. Also shown in Table 4 is the species in which each of the preferred target segments was found.

TABLE 4

Sequence and position of preferred target segments identified
in RANKL.

| | | | | |
|---|---|---|---|---|
| | TARGET | | REV COMP | |
| SITE | SEQ ID | TARGET | OF SEQ | SEQ ID |

| | | | | | | |
|---|---|---|---|---|---|---|
| ID | NO | SITE | SEQUENCE | ID | ACTIVE IN | NO |

| | | | | | | |
|---|---|---|---|---|---|---|
| 95966 | 11 | 20 | ctgccaggacctttgtgaac | 155 | M. musculus | 274 |
| 95968 | 11 | 52 | gggccgtggcggagtctgct | 157 | M. musculus | 275 |
| 95969 | 11 | 58 | tggcggagtctgctcggcgg | 158 | M. musculus | 276 |
| 95970 | 11 | 95 | gagagaacgatcgcggagca | 159 | M. musculus | 277 |
| 95971 | 11 | 99 | gaacgatcgcggagcagggc | 160 | M. musculus | 278 |
| 95973 | 11 | 137 | gcgccatgcgccgggccagc | 162 | M. musculus | 279 |
| 95974 | 11 | 140 | ccatgcgccgggccagccga | 163 | M. musculus | 280 |
| 95975 | 11 | 142 | atgcgccgggccagccgaga | 164 | M. musculus | 281 |
| 95977 | 11 | 262 | ccgccacccgccgcctcccg | 166 | M. musculus | 282 |
| 95978 | 11 | 270 | cgccgcctcccgctccatgt | 167 | M. musculus | 283 |
| 95979 | 11 | 271 | gccgcctcccgctccatgtt | 168 | M. musculus | 284 |
| 95980 | 11 | 340 | ctgttcctgtactttcgagc | 169 | M. musculus | 285 |
| 95981 | 11 | 448 | tcgactctggagagtgaaga | 170 | M. musculus | 286 |
| 95982 | 11 | 512 | gggccgtgcagaaggaactg | 171 | M. musculus | 287 |
| 95983 | 11 | 598 | gtggcccagcgaggcaagcc | 172 | M. musculus | 288 |
| 95985 | 11 | 783 | cctgtacgccaacatttgct | 173 | M. musculus | 289 |
| 95986 | 11 | 832 | cctacagactatcttcagct | 174 | M. musculus | 290 |
| 95987 | 11 | 867 | taaaaccagcatcaaaatcc | 175 | M. musculus | 291 |
| 95988 | 11 | 925 | aactggtcgggcaattctga | 176 | M. musculus | 292 |
| 95989 | 11 | 991 | gctggtgaagaaattagcat | 177 | M. musculus | 293 |
| 95990 | 11 | 1024 | ccttccctgctggatccgga | 178 | M. musculus | 294 |
| 95991 | 11 | 1092 | agactcatttcgtggaacat | 179 | M. musculus | 295 |
| 95992 | 11 | 1159 | tgtctatacatgtgtaagac | 180 | M. musculus | 296 |
| 95995 | 11 | 1337 | tgggagaggtattccgatgc | 183 | M. musculus | 297 |
| 95996 | 11 | 1411 | ccctggacatgtgccactga | 184 | M. musculus | 298 |
| 95997 | 11 | 1507 | acattgcgctgggacctgca | 185 | M. musculus | 299 |
| 95998 | 11 | 1513 | cgctgggacctgcaaataag | 186 | M. musculus | 300 |
| 96005 | 11 | 1738 | ccctgaaggtactcgtagct | 193 | M. musculus | 301 |
| 96006 | 11 | 1843 | aactatgcaagcaaaataaa | 194 | M. musculus | 302 |
| 96009 | 11 | 1980 | ggctactgaaatctgtcagg | 197 | M. musculus | 303 |
| 202604 | 18 | 15 | ccgagactacggcaagtacc | 202 | R. norvegicus | 304 |
| 202605 | 18 | 20 | actacggcaagtacctgcgc | 203 | R. norvegicus | 305 |
| 202606 | 18 | 178 | caggtggtctgcagcatcgc | 204 | R. norvegicus | 306 |
| 202607 | 18 | 183 | ggtctgcagcatcgctctgt | 205 | R. norvegicus | 307 |
| 202608 | 18 | 188 | gcagcatcgctctgttcctg | 206 | R. norvegicus | 308 |
| 202611 | 18 | 203 | tcctgtactttcgagcgcag | 209 | R. norvegicus | 309 |
| 202613 | 18 | 213 | tcgagcgcagatggatccta | 211 | R. norvegicus | 310 |
| 202614 | 18 | 218 | cgcagatggatcctaacaga | 212 | R. norvegicus | 311 |
| 202615 | 18 | 223 | atggatcctaacagaatatc | 213 | R. norvegicus | 312 |
| 202617 | 18 | 238 | atatcagaagacagcacgcg | 215 | R. norvegicus | 313 |
| 202619 | 18 | 268 | agaattctgagactccgtga | 217 | R. norvegicus | 314 |
| 202620 | 18 | 279 | actccgtgaaaatacaggtt | 218 | R. norvegicus | 315 |
| 202621 | 18 | 293 | caggtttgcaggactcgact | 219 | R. norvegicus | 316 |
| 202622 | 18 | 298 | ttgcaggactcgactctgga | 220 | R. norvegicus | 317 |
| 202623 | 18 | 369 | ctttcaaggggccgtgcaaa | 221 | R. norvegicus | 318 |
| 202625 | 18 | 397 | caacacattgtggggccaca | 223 | R. norvegicus | 319 |
| 202629 | 18 | 542 | ataaagtcagtctgtcctct | 227 | R. norvegicus | 320 |
| 202632 | 18 | 571 | gatcgaggctgggccaagat | 230 | R. norvegicus | 321 |
| 202633 | 18 | 576 | aggctgggccaagatctcta | 231 | R. norvegicus | 322 |
| 202635 | 18 | 601 | acgttaagcaacggaaaact | 233 | R. norvegicus | 323 |
| 202636 | 18 | 607 | agcaacggaaaactaagggt | 234 | R. norvegicus | 324 |
| 202637 | 18 | 612 | cggaaaactaagggttaacc | 235 | R. norvegicus | 325 |
| 202638 | 18 | 617 | aactaagggttaaccaagat | 236 | R. norvegicus | 326 |
| 202643 | 18 | 637 | ggcttctattacctgtacgc | 241 | R. norvegicus | 327 |
| 202644 | 18 | 642 | ctattacctgtacgccaaca | 242 | R. norvegicus | 328 |
| 202645 | 18 | 647 | acctgtacgccaacatttgc | 243 | R. norvegicus | 329 |
| 202647 | 18 | 703 | gactatcttcagctgatggt | 245 | R. norvegicus | 330 |
| 202648 | 18 | 717 | gatggtatatgtcgttaaaa | 246 | R. norvegicus | 331 |
| 202650 | 18 | 736 | accagcatcaaaatcccaag | 248 | R. norvegicus | 332 |
| 202657 | 18 | 911 | aagatgcgacgtactttggg | 255 | R. norvegicus | 333 |
| 202658 | 18 | 916 | gcgacgtactttggggcttt | 256 | R. norvegicus | 334 |
| 202659 | 18 | 921 | gtactttggggctttcaaag | 257 | R. norvegicus | 335 |
| 202660 | 18 | 938 | aagttcaagacatagactga | 258 | R. norvegicus | 336 |
| 202661 | 200 | 178 | gagccttagcatggatatgc | 259 | R. norvegicus | 337 |
| 202662 | 200 | 231 | tgtagatacatgtgtaagac | 260 | R. norvegicus | 338 |
| 202663 | 200 | 238 | acatgtgtaagactactaag | 261 | R. norvegicus | 339 |
| 202664 | 200 | 262 | gtggcccacgttgtacaaaa | 262 | R. norvegicus | 340 |
| 202665 | 200 | 293 | ctctcgaccctgtacaggtc | 263 | R. norvegicus | 341 |
| 202666 | 200 | 346 | tcatggtgatcacacaatgg | 264 | R. norvegicus | 342 |
| 202668 | 200 | 482 | aactcctgggcctgtgccac | 266 | R. norvegicus | 343 |
| 202669 | 200 | 491 | gcctgtgccactgagaacct | 267 | R. norvegicus | 344 |
| 202670 | 200 | 523 | ggtaccgttacattgcagag | 268 | R. norvegicus | 345 |
| 202671 | 200 | 535 | ttgcagagaaatgatggttt | 269 | R. norvegicus | 346 |
| 202672 | 200 | 588 | gctggaacctgcaaataagt | 270 | R. norvegicus | 347 |
| 95980 | 18 | 199 | ctgttcctgtactttcgagc | 169 | R. norvegicus | 285 |
| 95985 | 18 | 648 | cctgtacgccaacatttgct | 173 | R. norvegicus | 289 |
| 95987 | 18 | 732 | taaaaccagcatcaaaatcc | 175 | R. norvegicus | 291 |

As these “preferred target segments” have been found by experimentation to be open to, and accessible for, hybridization with the antisense compounds of the present invention, one of skill in the art will recognize or be able to ascertain, using no more than routine experimentation, further embodiments of the invention that encompass other compounds that specifically hybridize to these preferred target segments and consequently inhibit the expression of RANKL.

According to the present invention, antisense compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 18

Western Blot Analysis of RANKL Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to RANKL is used, with a radiolabeled or fluorescently labeled secondary antibody directed against the primary antibody species. Bands are visualized using a PHOSPHORIMAGER™ (Molecular Dynamics, Sunnyvale Calif.).

### Example 19

Antisense Inhibition of Mouse RANKL Expression by Chimeric Phosphorothioate Oligonucleotides having 2′-MOE Wings and a Deoxy Gap: Dose Response

In accordance with the present invention, a subset of the antisense oligonucleotides in Example 16 was further investigated in dose-response studies. The oligonucleotides used in this investigation were ISIS 180812 (SEQ ID NO: 178), ISIS 180814 (SEQ ID NO: 180), ISIS 180819 (SEQ ID NO: 185), ISIS 180828 (SEQ ID NO: 194) and the control oligonucleotide ISIS 101757 (AGGTGCTCAGGACTCCATTT, SEQ ID NO: 355). ISIS 101757 is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

C2C12 cells were treated with the oligonucleotides at 10, 50, 75, 100, and 150 nM as described in other examples herein. The compounds were analyzed for their effect on mouse RANKL mRNA levels in C2C12 cells by quantitative real-time PCR as described in other examples herein. The results are expressed as percent inhibition relative to the untreated control. The data, shown in Table 5, are averages from two experiments and illustrate the oligonucleotides of the present invention are able to downregulate RANKL mRNA expression in a dose-dependent manner.

TABLE 5
Inhibition of mouse RANKL mRNA levels by chimeric
phosphorothioate oligonucleotides having 2′-MOE wings and a
deoxy gap

| | ISIS # | | | | |
|---|---|---|---|---|---|
| | 180812 | 180814 | 180819 | 180828 | 101757 |
| Dose (nM) | % Inhibition of RANKL mRNA | | | | |
| 10 | 0 | 12 | 41 | 31 | N.D. |
| 50 | 5 | 14 | 39 | 43 | 17 |
| 75 | 13 | 61 | 57 | 23 | N.D. |
| 100 | 20 | 70 | 71 | 31 | 13 |
| 150 | 45 | 81 | 75 | 40 | N.D. |

### Example 20

Short-term Bone Resorption Model: RANKL mRNA Expression and Serum Calcium Levels after PTH Infusion

Parathyroidectomized rats are a well-established, short-term model of bone resorption and are useful in the investigation of antiresorptive agents. However, it is difficult to surgically remove only the parathyroid gland of rodents, and the inadvertent removal of the adjacent thyroid gland often has effects on thyroid hormone levels that introduce into the experiment an undesirable level of variability. Continuous infusion of parathyroid hormone (PTH) into intact, young (6-9 weeks old) mice reproduces the short-term bone resorption activity seen in parathyroidectomized rodents and is thus a useful, non-invasive model for the study of antiresorptive agents. The PTH infusion activates bone osteoclasts and causes them to degrade bone matrix, with a resulting rise in serum calcium. In accordance with the present invention, a short-term mouse model of bone resorption was developed and used to evaluate the effects of PTH infusion on serum calcium concentration and RANKL mRNA levels.

Female, Swiss-Webster mice (5-8 weeks old) were placed on a low calcium diet and implanted with mini-pumps delivering PTH continuously. The pumps were calibrated to deliver 1 ug PTH per 100 g bodyweight per 6 hour time period (1 ug/100 g/6 hr). With this procedure, over a 24 hour period, a total of 4 ug PTH per 100 g body weight is delivered (4 ug/100 g/24 hr). PTH infusion was conducted in these mice for 6, 12, 18 and 24 hours. At the end of each time period, the mice were sacrificed and measurements were made of the serum calcium, using the Sigma Diagnostics Calcium Kit (Sigma-Aldrich), and RANKL mRNA expression in the proximal tibia, as described in other examples herein. The data are expressed as percent increase relative to no PTH treatment. The results, shown in Table 6, are the average of three mice per time point and demonstrate that PTH infusion increases serum calcium concentration as much as 230% after 24 hours. The data also demonstrate that PTH induces a large increase in RANKL mRNA expression in proximal tibia, particularly at 6 hours of PTH treatment.

TABLE 6
Serum calcium concentration and RANKL mRNA in mice infused
with 4 ug/100 g PTH over 24 hours

| | Serum Calcium | |
|---|---|---|
| Time | Concentration | RANKL mRNA |
| hours | % control | % control |
| 0 | 100 | 100 |
| 6 | 109 | 787 |
| 12 | 115 | 242 |
| 18 | 125 | 117 |
| 24 | 230 | 205 |

### Example 21

RANKL mRNA Expression in Primary Mouse Osteoblast Cells after PTH Treatment: Time Course Study

In a further embodiment of this invention, the effect of PTH treatment on the expression of RANKL was determined in primary osteoblast cells isolated from mice skull caps. Primary osteoblastic cells were treated for 24 hours with either saline or the transfection reagent FuGENE 6 (Roche Applied Science, Indianapolis, Ind.). During the last 2 hours, the cells were treated with 10 nM PTH. The RNA was then harvested and the expression levels of RANKL mRNA were measured by the methods described herein. The data are expressed as percent increase over untreated control cells. The data are summarized in Table 7 and demonstrate that, in the presence of either saline or FuGENE 6, RANKL mRNA is upregulated in response to PTH, with peak expression occurring after two hours of PTH treatment.

TABLE 7
Effect of PTH treatment on RANKL mRNA expression in primary
osteoblast cells in the presence of saline or FuGENE 6

RANKL mRNA
% control

| Time | Saline | FuGENE 6 |
|---|---|---|
| (hours) | Treatment | Treatment |
| 0 | 100 | 100 |
| 0.5 | 168 | 185 |
| 1 | 700 | 750 |
| 2 | 908 | 1275 |
| 4 | 383 | 467 |
| 6 | 326 | 340 |

### Example 22

Antisense Inhibition of RANKL mRNA Expression Followed by PTH Treatment in Primary Mouse Osteoblast Cells: Dose Response

In a further embodiment of this invention, the effect of RANKL antisense oligonucleotides and PTH treatment on the expression of RANKL was determined in primary osteoblast cells isolated from mice skull caps.

ISIS 143624 (TGCTCAGCGAGTGTGCCAGC, SEQ ID NO: 348) was used as a control oligonucleotide. ISIS 143624 is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P=S) throughout the oligonucleotide. All cytidine residues are 5-methylcytidines.

Primary osteoblastic cells were transfected for 24 hours with either Isis 180819 (SEQ ID NO: 185) or the control oligo ISIS 143624 (SEQ ID NO: 348) at a dose of 63 mM, 125 nM, 250 nM, or 500 nM by the methods described herein but using the transfection reagent FuGENE 6 (Roche Applied Science, Indianapolis, Ind.). During the last 2 hours of oligonucleotide transfection, the cells were treated with 10 nM PTH. The RNA was then harvested and the expression levels of RANK mRNA were measured by the methods described herein. The results are expressed as percent inhibition relative to PTH treatment alone and represent the average from three experiments. The data are summarized in Table 8 and illustrate that the oligonucleotide of the present invention is able to inhibit the PTH-induced increase in RANKL mRNA expression, which was demonstrated in Example 21, in a dose dependent manner. The control oligonucleotide is unable to reduce RANKL mRNA levels.

TABLE 8
Antisense inhibition of RANKL mRNA expression followed by PTH
treatment in primary mouse osteoblast cells: dose response

| | % RANKL mRNA |
|---|---|
| | inhibition |
| PTH alone | 0 |
| Dose of | ISIS # |

| oligonucleotide | 180819 | 143624 |
|---|---|---|
| 63 nM | 19 | 4 |
| 125 nM | 39 | 3 |
| 250 nM | 71 | 0 |
| 500 nM | 85 | 9 |

### Example 23

Effects of Antisense Inhibition of RANKL after PTH Infusion in the Proximal Tibia and Calvaria in Mice: Dose Response

In accordance with the present invention, the levels of RANKL in the proximal tibia and calvaria in mice were measured following antisense oligonucleotide treatment and PTH infusion.

Female Swiss-Webster mice (5-8 wks old) fed a low-calcium diet were treated with ISIS 180819 (SEQ ID NO: 185) by subcutaneous injection at a dose of either 30 mg/kg or 50 mg/kg or intraperitoneal injection at a dose of either 15 mg/kg, 30 mg/kg or 50 mg/kg. After 2 weeks of daily treatment, the mice were infused with PTH at a dose of 1 ug/100 g/6 hr. After 24 hours of PTH infusion (total PTH delivery of 4 ug/100 g), the mice were sacrificed. The proximal tibia and calvaria were isolated and the level of RANKL mRNA expression was determined in each using two different primer-probe sets to confirm the results. The first primer-probe set was #1207, consisting of TTTATTCCATAAATGTTGGGGGATT (forward primer, SEQ ID NO: 349), TTGGACACCTGAATGCTAATTT (reverse primer, SEQ ID NO: 350) and TTCAAGCTCCGAGCTGGTGAAGA (probe, SEQ ID NO: 351). The second primer-probe set was #1015, consisting of CAACCCTTCCCTGCTGGA (forward primer, SEQ ID NO: 352), CAGTCTATGTCCTGAACTTTGAAAGC (reverse primer, SEQ ID NO: 353) and CCGGATCAAGATGCGACGTACTTTGG (probe, SEQ ID NO: 354). The results are expressed as percent inhibition relative to PTH treatment alone and represent the average of eight mice per group. The data are summarized in Table 9 and show that treatment of mice with the oligonucleotide of the present invention is capable of inhibiting the PTH-induced increase in RANKL mRNA expression (demonstrated in vivo in Example 20) in the proximal tibia and calvaria in a dose-dependent manner, with greater inhibition observed in the calvaria.

TABLE 9
Effect antisense inhibition of RANKL mRNA levels in the
proximal tibia and calvaria of mice following PTH treatment:
dose response and delivery method

| % Inhibition of RANKL | |
|---|---|
| Proximal tibia | Calvaria |
| Primer-Probe set | Primer-Probe set |

| | 1207 | 1015 | 1207 | 1015 |
|---|---|---|---|---|
| Oligonucleotide | | | | |
| Dose (mg/kg) | | | | |
| 0 | 0 | 0 | 0 | 0 |
| Subcutaneous | | | | |
| injection | | | | |
| 30 | 10 | 5 | 38 | 38 |
| 50 | 36 | 32 | 52 | 60 |
| Intraperitoneal | | | | |
| injection | | | | |
| 15 | 0 | 2 | N.D. | N.D. |
| 30 | 0 | 0 | 24 | 39 |
| 50 | 13 | 21 | 54 | 51 |

### Example 24

Serum Calcium Concentration 24 Hours after PTH Infusion in Mice Treated with RANKL Antisense Oligonucleotides: Dose Response

In accordance with the present invention, the effect of antisense oligonucleotide inhibition on PTH-induced serum calcium concentration increase was measured in mice. The serum calcium concentration in mice was measured as a function of antisense oligonucleotide dose. Calcitonin, a known bone antiresorptive agent, was used as a control for antiresorptive activity.

Female Swiss-Webster (5-8 wks old) mice fed a low-calcium diet were dosed subcutaneously for two weeks with either the oligonucleotides ISIS 180819 (SEQ ID NO: 185) or 180814 (SEQ ID NO: 180) at concentrations of 5 mg/kg, 15 mg/kg, 30 mg/kg, or 40 mg/kg. A control group of mice were treated with calcitonin (400 ng) plus PTH (1 ug/100 g/6 hr), PTH alone (1 ug/100 g/6 hr) or saline. Treatment with oligonucleotides was followed with infusion of PTH by subcutaneously implanted mini-pumps at a dose of 1 ug/100 g/6 hr. After 24 hours of PTH infusion (total PTH delivery of 4 ug/100 g), the mice were sacrificed and the serum calcium concentration was measured with the Sigma Diagnostics Calcium Kit (Sigma-Aldrich). The data, summarized in Table 10 are averages from eight mice. Percent mRNA inhibition is normalized to PTH alone treatment. The data illustrate that the oligonucleotides of the present invention can, in a dose-dependent manner, lessen the PTH-induced increase in serum calcium concentration.

TABLE 10
Serum calcium concentration 24 hours after PTH infusion in
mice treated with RANKL antisense oligonucleotides for two
weeks: dose response

| ISIS | | ISIS | |
|---|---|---|---|
| 180819 | | 180814 | |
| Serum | RANKL | Serum | RANKL |
| Calcium, | mRNA, | Calcium, | mRNA, |
| mg/dl | % inhib | mg/dl | % inhib |

No oligonucleotide treatment

| | | | | |
|---|---|---|---|---|
| Saline | 10 | 58 | 10 | 53 |
| PTH + Calcitonin | 14 | 0 | 13 | 0 |
| PTH | 22 | 0 | 22 | 0 |

Isis oligonucleotide-treated

| | | | | |
|---|---|---|---|---|
| 5 mg/kg | 21 | 0 | 22 | 0 |
| 15 mg/kg | 17 | 0 | 20 | 0 |
| 30 mg/kg | 19 | 0 | 18 | 27 |
| 40 mg/kg | N.D. | N.D. | 19 | 6 |

As illustrated in this and other examples herein, the inhibition of PTH-induced serum calcium concentration by RANKL antisense oligonucleotides (35% inhibition, n=8) is effective, although less so than inhibition by calcitonin, a known antiresorptive agent (66% inhibition, n=8). If desired, additional antisense oligonucleotides can be screened in a like manner to identify those with more or less inhibitory properties. These examples also illustrate that the PTH infusion model of the present invention provides a useful tool for the study of antiresorptive agents, particularly antisense oligonucleotides.

### Example 25

Serum Calcium Concentration in Mice Treated with RANK Antisense Oligonucleotide: Dose Response and Delivery Method Comparison

In accordance with the present invention, antisense inhibition of the RANKL receptor, RANK, was evaluated in the short-term model of bone resorption. Serum calcium concentration was measured in mice treated with RANK antisense oligonucleotides at different doses, delivered by subcutaneous mini-pumps or subcutaneous injection.

ISIS 181071 (TCCTCGAGAGGTCTCCTTGC, SEQ ID NO: 356) is an antisense oligonucleotide targeted to mouse RANK. ISIS 181071 is a chimeric oligonucleotide (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

Female Swiss-Webster mice (5-8 wks old), fed a low-calcium diet were treated with ISIS 181071 (SEQ ID NO: 356) for 15 days. The dose of oligonucleotide was either 30 mg/kg, 20 mg/kg, 10 mg/kg, 3 mg/kg, or 0.1 mg/kg administered by daily subcutaneous injections for the first five days, followed by mini-pump infusion for the remaining 10 days, or the dose was 10 mg/kg or 30 mg/kg administered daily by subcutaneous injection for the entire 15 day period. No PTH was administered to the mice that received oligonucleotide. A group of control mice were treated with either saline, PTH alone (1 ug/100 g/6 hr) or calcitonin (400 ng) plus PTH (1 ug/100 g/6 hr). After 16 hours (total PTH delivery of 3 ug/100 g), blood samples were taken from the tail of the mice and the serum calcium concentration was measured with the Sigma Diagnostics Calcium Kit (Sigma-Aldrich). The data are summarized in Table 11 and demonstrate that, with no PTH dose, the serum calcium concentration is similar with all of oligonucleotide doses and delivery methods tested. Thus, increase in serum calcium concentration is due to the PTH treatment rather than the oligonucleotide treatment.

TABLE 11
Serum calcium concentration in mice treated with RANK
antisense oligonucleotide: dose response and delivery method
comparison

| | Serum calcium |
|---|---|
| | concentration (mg/dL) |
| saline | 9 |
| PTH | 9 |
| PTH + Calcitonin | 8 |

ISIS 181071 injection and infusion

| | |
|---|---|
| 30 mg/kg | 9 |
| 20 mg/kg | 9 |
| 10 mg/kg | 9 |
| 3 mg/kg | 9 |
| 0.1 mg/kg | 9 |

ISIS 181071 injection

| | |
|---|---|
| 30 mg/kg | 8 |
| 10 mg/kg | 9 |

### Example 26

Serum Calcium Concentration and RANK mRNA Expression in Mouse Proximal Tibia after Antisense Oligonucleotide Treatment and PTH Infusion: Dose Response Study and Delivery Method Comparison

In accordance with the present invention, ISIS 181071 was evaluated for its ability to inhibit RANK expression and PTH-induced serum calcium concentration increase following 18 days of oligonucleotide treatment.

Female Swiss-Webster mice (5-8 wks old), fed a low-calcium diet, were treated for 18 days with ISIS 181071 (SEQ ID NO: 356) at different doses and through different delivery methods. The dose of oligonucleotide was either 30 mg/kg, 20 mg/kg, 10 mg/kg, 3 mg/kg, or 0.1 mg/kg administered by daily subcutaneous injections for the first five days, followed by mini-pump infusion for the remaining 13 days, or the dose was 10 mg/kg or 30 mg/kg administered daily by subcutaneous injection for the entire 18 day period. After 18 days of treatment with oligonucleotides, the mice were infused with PTH by subcutaneously implanted mini-pumps at a dose of 1 ug/100 g/6 hr. A group of control mice received saline, calcitonin (400 ng) plus PTH (1 ug/100 g/6 hr) or PTH alone (1 ug/100 g/6 hr). After 24 hours of PTH infusion (total PTH delivery of 4ug/100 g), the mice were sacrificed and measurements were made of serum calcium with the Sigma Diagnostics Calcium Kit (Sigma-Aldrich) and RANK mRNA expression in proximal tibia (as described in other examples herein). The results are shown in Table 12 are normalized to PTH treatment alone and are the average of four mice per group. The data illustrate that the tested methods of delivering oligonucleotides can inhibit the expression of

RANK mRNA and alleviate the rise in PTH-induced serum calcium concentration in a dose-dependent manner.

TABLE 12
Serum calcium concentration and RANK mRNA expression in
proximal tibia after PTH infusion in mice: dose response
study and delivery method comparison

| | RANK mRNA | serum |
|---|---|---|
| | % inhibition | calcium |
| | (normalized to PTH) | (mg/dl) |
| Saline | 47 | 11 |
| PTH | 0 | 20 |
| PTH + Calcitonin | 15 | 14 |

ISIS 181071 Dosed by injection and infusion

| | | |
|---|---|---|
| 30 mg/kg | 32 | 17 |
| 20 mg/kg | 33 | 18 |
| 10 mg/kg | 23 | 18 |
| 3 mg/kg | 5 | 20 |
| 0.1 mg/kg | 19 | 21 |

ISIS 181071 Dosed by injection

| | | |
|---|---|---|
| 30 mg/kg | 30 | 14 |
| 10 mg/kg | 29 | 18 |

### Example 27

Serum Calcium Concentration and RANK mRNA Expression in Mouse Proximal Tibia after Antisense Oligonucleotide Treatment and PTH Infusion: Time Course Study

In accordance with the present invention, ISIS 181071 was administered to mice for different time periods to evaluate its ability to inhibit RANK expression and PTH-induced serum calcium concentration increase as a function of oligonucleotide treatment time.

Female Swiss-Webster mice (5-8 wks old) were fed a low-calcium diet and received daily subcutaneous injections of 30 mg/kg of ISIS 181071 (SEQ ID NO: 356) for 2, 3, 5, 7, 10, 14, or 21 days. At the end of the oligonucleotide treatment period, mice were infused with PTH by subcutaneously implanted mini-pumps at a dose of 1 ug/100 g/6 hr. A group of control mice were dosed with either saline, PTH alone (1 ug/100 g/6 hr) or calcitonin (400 ug) plus PTH (1 ug/100 g/6 hr). Following 24 hours of PTH infusion (total PTH delivery of 4ug/100 g), the mice were sacrificed and measurements were made of serum calcium with the Sigma Diagnostics Calcium Kit (Sigma-Aldrich) and RANK mRNA expression in the proximal tibia (as described in other examples herein). Percent RANK mRNA inhibition is normalized to PTH alone treatment. The data are the average of four mice per group and are summarized in Table 13. The data demonstrate that treatment with the antisense oligonucleotide of the present invention lessens the PTH-induced increase in serum calcium concentration, and that this effect can be correlated with the inhibition of RANK mRNA in the proximal tibia.

TABLE 13
Serum calcium and RANK mRNA expression following antisense
oligonucleotide and PTH infusion in mice: time course study

| | |
|---|---|
| | RANK mRNA |
| | % inhibition |
| Serum calcium | (normalized to PTH) |

| | concentration | Proximal | Bone |
|---|---|---|---|
| | (mg/dL) | Tibia | Marrow |
| Saline | 11 | 59 | 55 |
| PTH alone | 21 | 0 | 0 |
| PTH + Calcitonin | 15 | 35 | 1 |

ISIS 181071 oligonucleotide treated

| | | | |
|---|---|---|---|
| 21 days | 15 | 52 | 8 |
| 14 days | 15 | 53 | 36 |
| 10 days | 18 | 37 | 18 |
| 7 days | 16 | 61 | 44 |
| 5 days | 16 | 32 | 32 |
| 3 days | 17 | 14 | 35 |
| 2 days | 20 | 34 | 0 |

### Example 28

Serum Calcium Concentration after PTH Infusion in Mice Treated with RANK Antisense Oligonucleotide: Dosing Schedule Study

In accordance with the present invention, various antisense oligonucleotide dosing schedules were tested for their ability to inhibit serum calcium concentration increase resulting from PTH infusion.

Female Swiss-Webster mice (5-8 wks old) were fed a low-calcium diet. The mice were dosed with the same total amount of ISIS 181071 (SEQ ID NO: 356) (450 mg/kg), however the frequency of the dosage was varied. The first group received daily injections for 2 weeks. The second group received daily injections of 30 mg/kg/day for 5 days; the remaining 300 mg/kg was divided into 12 injections over 24 days and were administered every other day. The third group received daily injections of 30 mg/kg/day for 5 days; the remaining 300 mg/kg was divided into 8 injections over 24 days and were administered every third day. The fourth group received daily injections of 30 mg/kg/day for 5 days; the remaining 300 mg/kg was divided into 6 injections over 24 days and were administered every fourth day. Each group contained eight mice. Treatment with oligonucleotides was followed with infusion of PTH by subcutaneously implanted mini-pumps at a dose of 1 ug/100 g/6 hr. A control group received saline or PTH alone (1 ug/100 g/6 hr). After 24 hours of PTH infusion (total PTH delivery of 4 ug/100 g), the mice were sacrificed and the serum calcium concentration was measured with the Sigma Diagnostics Calcium Kit (Sigma-Aldrich). The data are shown in Table 14 and are the average of eight mice per group. The data demonstrate that the various dosing schedules of the oligonucleotide of the present invention can similarly prevent the rise in serum calcium concentration that occurs in response to PTH infusion.

TABLE 14
Serum calcium concentration after PTH infusion in mice
treated with RANK antisense oligonucleotide:
dosing schedule study

| | serum calcium |
|---|---|
| | concentration (mg/dL) |
| Saline | 9 |
| PTH | 21 |

injection schedule of ISIS 181071

| | |
|---|---|
| daily | 16 |
| every other day | 16 |
| every third day | 17 |
| every fourth day | 18 |

### Example 29

Serum Calcium Concentration and Antisense Inhibition of RANK mRNA Expression in Proximal Tibia after PTH Infusion in Mice: Specificity and Dose Response

In accordance with the present invention, the specificity of RANK inhibition by a RANK antisense oligonucleotide was compared to that of an 8-base pair mismatch oligonucleotide.

ISIS 181080 (GAGCTCCCGGACCCTGAGGC, SEQ ID NO: 357) is an antisense oligonucleotide targeted to mouse RANK. ISIS 208565 (TCCTCGAGAGGTCTCCTTGC, SEQ ID NO: 358) is an 8-base pair mismatch oligonucleotide. ISIS 181071 and ISIS 208565 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE)nucleotides. The internucleoside (backbone) linkages are phosphorothioate (P═S) throughout the oligonucleotide. All cytidine residues are 5-methylcytidines.

Female Swiss-Webster mice (5-8 wks old) fed a low-calcium diet were treated for two weeks with either ISIS 181080 or ISIS 208565. The dose of oligonucleotide was either 40 mg/kg, 30 mg/kg, 20 mg/kg, or 10 mg/kg. After the 2-week treatment with oligonucleotides, the mice were infused with PTH by subcutaneously implanted mini-pumps at a dose of 4 ug/100 g over 24 hours. A group of control mice were dosed with either saline, PTH alone (1 ug/100 g/6 hr) or calcitonin (400 ng) plus PTH (1 ug/100 g/6 hr). After 24 hours of PTH infusion (total PTH delivery of 4 ug/100 g), the mice were sacrificed and measurements were made of serum calcium with the Sigma Diagnostics Calcium Kit (Sigma-Aldrich) and RANK mRNA expression in proximal tibia (as described in other examples herein). The results shown in Table 15 are the average from four mice per group. Percent inhibition of RANK mRNA is normalized to PTH treatment alone. The data demonstrate that the oligonucleotide of the present invention inhibits expression of RANK mRNA in vivo and consequently lessens the PTH-induced rise in serum calcium concentration. The data also illustrate that these effects are occurring due to an antisense mechanism.

TABLE 15
Serum calcium concentration and antisense inhibition of RANK
mRNA expression in proximal tibia after PTH infusion:
specificity and dose response

| | RANK mRNA | serum |
|---|---|---|
| | % inhibition | calcium |
| | (normalized to PTH) | (mg/dl) |
| Saline | 63 | 10 |
| PTH | 0 | 20 |
| PTH + Calcitonin | 2 | 15 |

ISIS 181080

| | | |
|---|---|---|
| 10 mg/kg | 47 | 23 |
| 20 mg/kg | 38 | 17 |
| 30 mg/kg | 48 | 15 |
| 40 mg/kg | 65 | 16 |

ISIS 208565

| | | |
|---|---|---|
| 10 mg/kg | 0 | 21 |
| 20 mg/kg | 0 | 17 |
| 30 mg/kg | 19 | 18 |
| 40 mg/kg | 7 | 16 |

As illustrated in this and other examples herein, the inhibition of PTH-induced serum calcium concentration by RANKL antisense oligonucleotides (35% inhibition, n=8) is effective, although less so than inhibition by calcitonin, a known antiresorptive agent (66% inhibition, n=8). If desired, additional antisense oligonucleotides can be screened in a like manner to identify those with more or less inhibitory properies. These examples also illustrate that the PTH infusion model of the present invention provides a useful tool for the study of antiresorptive agents, particularly antisence oligonucleotides.

All references, issued patents and patent applications cited within the body of the instant specification are hereby incorporated by reference in their entirety, for all purposes.

1. A mouse model for bone metabolism, the model comprising a mouse exposed to a compound selected from the group consisting of parathyroid hormone (PTH), an analogue of PTH, and a fragment of PTH for a time sufficient whereby serum calcium concentration and RANKL mRNA expression are increased in the model.

2. The mouse model of claim 1, wherein the compound is PTH.

3. The mouse model of claim 1, wherein the mouse is exposed to the compound for about 0.5 h to about 96 h.

4. The mouse model of claim 3, wherein the mouse is exposed to the compound for about 24 h.

5. The mouse model of claim 1, wherein the mouse is exposed to about 0.5 ug to about 8 ug of the compound per 100 g of bodyweight.

6. The mouse model of claim 1, wherein the calcium concentration is increased by about 10%.

7. The mouse model of claim 6, wherein the calcium concentration is increased by about 25%.

8. The mouse model of claim 7, wherein the calcium concentration is increased by about 100% 24 h after exposure to the compound.

9. The mouse model of claim 1, wherein RANKL mRNA expression is increased by about 10%.

10. The mouse model of claim 1, wherein bone metabolism disease is osteoporosis.

11. A method of screening for a potentially therapeutic agent which affects bone metabolism, the method comprising:
administering the agent to the mouse model of claim 1; and
assessing the mouse for an alteration in a bone metabolism related marker affected by the agent.

12. A method for assessing the activity of potentially therapeutic agents useful for the treatment and prevention of osteoporosis, the method comprising:
providing a mouse model of claim 1;
administering the agent to the mouse model; and
assessing the affect of the agent on the mouse model treated with the agent compared to an untreated mouse model.

13. A method for testing a mouse model for bone metabolism disease, the method comprising:
administering to the mouse an antisense oligonucleotide to RANK or RANKL;
administering to the mouse a compound selected from the group consisting of parathyroid hormone (PTH), an analogue of PTH, and a fragment of PTH; and
assessing the affect of the antisense oligonucleotide on the mouse compared to a control mouse not treated with the antisense oligonucleotide.

14. The method of claim 13, wherein the antisense oligonucleotide is administered for about 1 day to about 30 days.

15. The method of claim 14, wherein the antisense oligonucleotide is administered from about 5 days to about 20 days.

16. The method of claim 13, wherein the antisense oligonucleotide is administered at a dose of about 5 mg/kg/day to about 100 mg/kg/day.

17. The method of claim 13, wherein the compound is administered after the complete administration of the antisense oligonucleotide.

18. The method of claim 17, wherein the compound is PTH.

19. The method of claim 17, wherein the mouse is exposed to the compound for about 0.5 h to about 96 h.

20. The method of claim 19, wherein the mouse is exposed to the compound for about 24 h.

21. The method of claim 17, wherein the mouse is exposed to about 0.5 ug to about 8 ug of the compound per 100 g of bodyweight.

22. The method of claim 13, wherein the antisense oligonucleotide is selected from the group consisting of SEQ ID No.: 180, SEQ ID No.: 185, SEQ ID No.: 356, and SEQ ID No.: 357, or combinations thereof.

23. The method of claim 13, further comprising a calcitonin treated mouse as a control.

24. The method of claim 13, wherein the antisense oligonucleotide modulates RANKL mRNA expression, RANK mRNA expression, or serum calcium concentration, and combinations thereof, compared to the control.

25. The method of claim 24, wherein the modulation is at least about 10%.
